(19) United States
(12) Patent Application Publication
(10) Pub. No.: US 2008/0319024 A1
(43) Pub. Date: Dec. 25, 2008

(54) Rosiglitazone Phosphate and Polymorphic Forms

(76) Inventors: Julia Greil, Kramsach (AT); Johannes Ludescher, Breitenbach (AT); Siegfried Wolf, Brixiegg (AT)

Correspondence Address: SANDOZ INC, 506 CARNEFIE CENTER, PRINCETON, NJ, 08540, US

(21) Appl. No.: 10/588,614

(22) PCT Filed: Feb. 11, 2005
(86) PCT No.: PCT/EP2005/001378

§ 371 Date: Jan. 24, 2007

(30) Foreign Application Priority Data
Feb. 13, 2004 (GB) 0403254.6
Dec. 14, 2004 (GB) 0427379.3

(51) Int. Cl. A61K 31/4439; C07D 417/12; A61P 3/04

(52) U.S. Cl. 514/342; 546/269.7

(57) ABSTRACT

The present invention relates to 5-[[4-[2-(methyl-2-pyridinylamino)ethoxy]phenyl]methyl]-2,4-thiazolidinedione phosphate, a novel salt of rosiglitazone and to novel polymorphic forms thereof. The invention is also directed to processes for preparation of rosiglitazone phosphate and its polymorphs. The compounds of the invention are useful for treatment and/or prophylaxis of diabetes mellitus, conditions associated with diabetes mellitus and certain complications thereof.

### FIELD OF THE INVENTION

This invention relates to a novel compound, to a process for the preparation of said compound, to pharmaceutical compositions containing said compound and to the use of such a compound and of such compositions in medicine. Additionally, the invention includes different polymorphic forms of said novel compound.

### BACKGROUND OF THE INVENTION

European Patent Application, Publication Number 0306228 relates to certain thiazolidinedione derivatives disclosed as having hypoglycaemic and hypolipidaemic activity. The compound of example 30 of EP-A-0306228 is 5-[[4-[2-(methyl-2-pyridinylamino) ethoxy]phenyl]methyl]-2,4-thiazolidinedione (according to Merck Index/13th Edition, Monograph number 8346, CAS Registry number: 122320-73-4), i.e. rosiglitazone.

International Application, Publication Number WO 94/05659 discloses certain salts of the compounds of EP-A-0306228. The preferred salt of WO 94/05659 is the maleic acid salt.

There remains a need for alternative salt forms which are straightforward to prepare and which have properties suitable for pharmaceutical processing on a commercial scale.

### DESCRIPTION OF THE INVENTION

The present inventors have now prepared and characterised a phosphoric acid salt of 5-[[4-[2-(methyl-2-pyridinylamino)ethoxy]phenyl]methyl]-2,4-thiazolidinedione, being a of 5-[[4-[2-(methyl-2-pyridinylamino)ethoxy]phenyl]methyl]-2,4-thiazolidinedione phosphate, hereinafter also referred to as the “Phosphate”, and have discovered that the “Phosphate” is particularly stable and hence is suitable for bulk preparation and handling.

Additionally, the inventors have prepared and characterized different polymorphic forms of the “Phosphate”, namely the polymorphic forms A, B, B1, C, D and E. Polymorphism is commonly defined as the ability of a substance, e.g. of a pharmaceutically active substance, to have two or more different crystal structures. Such different crystal forms are individually referred to as polymorphs. Said substances may also encapsulate solvent molecules when crystallized, these solvates or hydrates being referred to as pseudopolymorphs. Different polymorphs, pseudopolymorphs or the amorphous form of a given substance may differ from each other with respect to one or more physical properties such as melting point, solubility and dissociation, true density, crystal shape, compaction behaviour, flow properties, and/or solid state stability. These may appreciably influence pharmaceutical properties such as dissolution rate and/or bioavailability. It is also economically desirable that a given substance is stable for extended periods of time without the need of specialized storage conditions. It is therefore important to evaluate polymorphic forms of pharmaceutically active substances. The term “polymorphic forms” as herein used is understood to include both polymorphs and pseudopolymorphs of the compound of the invention, i.e. the Phosphate. Additionally the terms “polymorphic forms”, “Forms”, “polymorphs”, “crystalline polymorphs” and “crystalline polymorphic forms” as used herein are understood to have the same meaning and to be interchangeable.

5-[[4-[2-(methyl-2-pyridinylamino)ethoxy]phenyl]methyl]-2,4-thiazolidinedione as herein used is understood to mean rosiglitazone, in the form of a free base.

The novel Phosphate, and the polymorphic forms thereof, have also useful pharmaceutical properties and may be used for the treatment and/or prophylaxis of diabetes mellitus, conditions associated with diabetes mellitus and certain complications thereof.

In one aspect therefore, the present invention provides a salt of 5-[[4-[2-(methyl-2-pyridinylamino)ethoxy]phenyl]methyl]-2,4-thiazolidinedione and phosphoric acid, or a solvate or a non-solvated form thereof.

In an additional aspect, the present invention provides novel polymorphic forms of the novel phosphoric acid salt of 5-[[4-[2-(methyl-2-pyridinylamino)ethoxy]phenyl]methyl]-2,4-thiazolidinedione which are designated herein as Forms A, B, B1, C, D or E and which may be in the form of a solvate (Forms A, C and D), e.g. of a hydrate (Form A and C), or of a solvate with methanol (Form D), or in a non-solvated form, e.g. in the form of an anhydrate (Forms B, B1 and E).

The term “non-solvated form” as herein used is understood to mean being essentially free of residual inorganic or organic solvent media, e.g. being an anhydrous form.

Phosphoric acid is a triacid, so that the phosphate salts may theoretically exist in more than one stoichiometry. However, the inventors have isolated the Phosphate so far only in the form in which the molar ratio of 5-[[4-[2-(methyl-2-pyridinylamino)ethoxy]phenyl]methyl]-2,4-thiazolidinedione to phosphoric acid is or is approximately 1:1, which encompasses molar ratios from 1:0.9 to 1:1.2. Theoretically, the molar ratio of 5-[[4-[2-(methyl-2-pyridinylamino)ethoxy]phenyl]methyl]-2,4-thiazolidinedione to phosphoric acid could also be 3:1 or 2:1. Such molar ratios are also encompassed by the present invention.

Accordingly, in a further aspect the present invention provides a 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, or a solvate or a non-solvated form thereof.

Additionally, the present invention provides 5-[[4-[2-(methyl-2-pyridinylamino) ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, or a solvate or a non-solvated form thereof, and the polymorphic forms A, B, B1, C, D and E thereof. 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, is hereinafter also called “5-[[4-[2-(methyl-2-pyridinylamino)ethoxy]phenyl]methyl]-2,4-thiazolidinedione phosphate (1:1)”.

Preferably, the “Phosphate” is a hydrate or an anhydrate. More preferably, the “Phosphate” is in its polymorphic Form A, B, B₁ or E.

The Phosphate and its polymorphic forms may exist in one of several tautomeric forms, all of which are encompassed by the present invention. It will be appreciated that the present invention encompasses all of the isomeric forms of the Phosphate, preferably as a hydrate, including any stereoisomeric forms thereof, whether as individual isomers or as mixtures of isomers.

Without wishing to be bound by any particular mechanism or theory, the present applicants believe that in the 1:1 salt the phosphate anion may be associated with a proton (hydrogen atom) in addition to 5-[[4-[2-(methyl-2-pyridinylamino)ethoxy]phenyl]methyl]-2,4-thiazolidinedione, or may be associated with another cation, for example an alkali metal or ammonium cation. In this case, the salt may be described as a mixed salt.

As indicated above, the preferred aspect of the invention is a hydrate or an anhydrate of the Phosphate which hereinafter is also referred to as “Phosphate Hydrate” or “Phosphate Anhydrate”, respectively. Said Phosphate Hydrate exists in the polymorphic forms A or C, whereof Form A is preferred, and said Phosphate Anhydrate exists in the polymorphic forms B, B₁ or E, depending on the way of preparation and/or on the corresponding starting materials used as described below.

Thus, in one aspect, the present invention provides a crystalline polymorphic form of 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, in the form of a hydrate, herein designated as Form A, characterised by an X-ray powder diffraction (XRPD) pattern having intensity peaks at values expressed in 2-theta degrees of about 15.63, 15.75, 17.30, 19.61 and 21.47. Crystalline polymorphic Form A may further present intensity peaks at any one or more values selected from the following values expressed in 2-theta degrees: about 4.28, 5.38, 8.61, 9.92, 12.44, 14.04, 16.91, 21.66, 22.54, 24.10, 24.43, 24.77, 25.06, 25.81 and 26.28.

In another aspect, crystalline polymorph A is characterised by an X-ray powder diffraction (XRPD) pattern substantially in accordance with Table 1 and FIG. 1.

TABLE 1
X-Ray Powder Diffraction (XRPD) pattern of Form A showing
interplanar spacings
(d, given in Å, i.e. Angstroem), characteristic XRPD angles
(2 theta°) and relative intensities (in %)

| d-value | Angle | Rel. Intensity |
|---|---|---|
| (Å) | 2 theta° | (%) |
| 22.66 | 3.90 | 13 |
| 20.63 | 4.28 | 21 |
| 16.42 | 5.38 | 18 |
| 14.20 | 6.22 | 7 |
| 10.51 | 8.41 | 16 |
| 10.26 | 8.61 | 19 |
| 9.879 | 8.94 | 7 |
| 8.911 | 9.92 | 18 |
| 8.170 | 10.82 | 6 |
| 7.514 | 11.77 | 7 |
| 7.111 | 12.44 | 24 |
| 6.828 | 12.96 | 10 |
| 6.748 | 13.11 | 9 |
| 6.497 | 13.62 | 13 |
| 6.301 | 14.04 | 31 |
| 5.667 | 15.63 | 65 |
| 5.622 | 15.75 | 100 |
| 5.514 | 16.06 | 16 |
| 5.239 | 16.91 | 19 |
| 5.123 | 17.30 | 42 |
| 4.924 | 18.00 | 17 |
| 4.855 | 18.26 | 9 |
| 4.663 | 19.02 | 15 |
| 4.524 | 19.61 | 35 |
| 4.342 | 20.44 | 14 |
| 4.135 | 21.47 | 40 |
| 4.100 | 21.66 | 33 |
| 4.037 | 22.00 | 16 |
| 3.941 | 22.54 | 30 |
| 3.876 | 22.93 | 12 |
| 3.817 | 23.29 | 13 |
| 3.803 | 23.37 | 15 |
| 3.777 | 23.54 | 16 |
| 3.741 | 23.77 | 15 |
| 3.690 | 24.10 | 18 |
| 3.641 | 24.43 | 18 |
| 3.591 | 24.77 | 18 |
| 3.550 | 25.06 | 18 |
| 3.449 | 25.81 | 19 |
| 3.389 | 26.28 | 23 |
| 3.279 | 27.17 | 8 |
| 3.227 | 27.62 | 14 |
| 3.201 | 27.85 | 14 |
| 3.128 | 28.51 | 9 |
| 3.066 | 29.11 | 10 |
| 3.025 | 29.51 | 14 |
| 2.957 | 30.20 | 9 |
| 2.922 | 30.57 | 12 |
| 2.910 | 30.70 | 13 |
| 2.829 | 31.60 | 10 |
| 2.807 | 31.86 | 9 |
| 2.774 | 32.25 | 9 |
| 2.759 | 32.42 | 9 |
| 2.711 | 33.01 | 7 |
| 2.674 | 33.49 | 7 |
| 2.617 | 34.24 | 10 |
| 2.608 | 34.36 | 11 |
| 2.568 | 34.91 | 8 |
| 2.556 | 35.08 | 8 |
| 2.452 | 36.61 | 7 |
| 2.421 | 37.11 | 7 |
| 2.367 | 37.98 | 6 |
| 2.330 | 38.60 | 7 |
| 2.302 | 39.10 | 8 |
| 2.273 | 39.62 | 8 |

Optionally, crystalline polymorph A is additionally characterized by an infrared spectrum with bands observed at 2704, 1748, 1701, 1643, 1611, 1546, 1513, 1469, 1420, 1391, 1330, 1302, 1244, 1110, 1028, 928, 821, 767, 716 cm⁻¹, as depicted in FIG. 2. Form A may thus provide an infrared spectrum substantially in accordance with FIG. 2.

The Infrared absorption spectrum of the herein described polymorphic forms of the Phosphate is measured using a BRUKER FTIR-Tensor 27.

X-Ray Powder Diffraction (XRPD) pattern as herein shown is measured using a X-Ray Powder Diffractometer D-8 (AXS-BRUKER) and copper radiation with a 2-theta accuracy of sample data of ±0.05 degrees as described below:

Form A has a melting point in the range of 171-177° C. according to the method of Kofler (e.g. as described in Vogel, A. I., Practical Organic Chemistry, 3^{rd} edition, p. 82).

Form A is thus the Phosphate in which the ratio of 5-[[4-[2-(methyl-2-pyridinylamino) ethoxy]phenyl]methyl]-2,4-thiazolidinedione to phosphoric acid is (by mole) 1:1 which has been isolated as a Phosphate Hydrate containing approximately 0.1%-4.5%, e.g. approximately 0.8%-4%, e.g. preferably 1.6%-3.6% by weight water.

A particular example of Form A contains approximately 0.87% of water, consistent with a 1:0.23 hydrate. Further particular examples contain approximately 1.6% of water, consistent with a 1:0.42 hydrate, or approximately 2.3% of water, consistent with a 1:0.60 hydrate, or 3.3% of water, consistent with a 1:0.79 hydrate, or 3.58% of water, consistent with a 1:0.94 hydrate. All percentages are by weight.

Drying Form A e.g. at room temperature results in an approximately 1:0.4 hydrate; drying with the aid of a strong desiccant, e.g. P₂O₅, at about 45° C. results in an approximately 1:0.3 hydrate, and optional further drying at elevated temperatures, e.g. 70° C.-100° C., preferably 80° C., may lead to a water content of less than 0.1% by weight.

Room temperature as used herein is understood to mean temperatures of about 20° C. to about 35° C., e.g. of about 25° C. to about 28° C.

Exposure of Form A to high humidity results in an approximately 1:1 hydrate.

Accordingly, the crystalline polymorphic form of 5-[[4-[2-(methyl-2-pyridinylamino)ethoxy]phenyl]methyl]-2,4-thiazolidinedione phosphate (1:1), in the form of a hydrate, herein designated as Form A contains approximately up to 4.5% by weight water, e.g. contains approximately 3.6% water by weight being consistent to a 1:0.94 hydrate, or e.g. contains approximately 1.6% water by weight being consistent to a 10.42 hydrate.

In another aspect, the present invention provides a crystalline polymorphic form of 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, in the form of a hydrate, herein designated as Form C, characterized by an X-ray powder diffraction (XRPD) pattern having intensity peaks at values expressed in 2-theta degrees of about 12.86, 15.98, 16.26, 21.60 and 24.50. Crystalline polymorphic Form C may further present intensity peaks at any one or more values selected from the following values expressed in 2-theta degrees: about 11.32, 14.50, 16.47, 18.91, 19.99, 20.30, 23.45, 24.34 and 29.40.

The water content of Form C may lie in the range of 3.8 to 3.9% by weight.

In another aspect, crystalline polymorph C is characterised by an X-ray powder diffraction (XRPD) pattern substantially in accordance with Table 2 and FIG. 3.

TABLE 2
X-Ray Powder Diffraction (XRPD) pattern of Form C showing
interplanar spacings
(d, given in Å, i.e. Angstroem), characteristic XRPD angles
(2 theta°) and relative intensities (in %)

| d-value | Angle | Rel. Intensity |
|---|---|---|
| (Å) | 2 theta° | (%) |
| 22.18 | 3.98 | 20 |
| 10.93 | 8.08 | 5 |
| 8.842 | 10.00 | 4 |
| 7.809 | 11.32 | 38 |
| 7.294 | 12.12 | 20 |
| 6.876 | 12.86 | 62 |
| 6.104 | 14.50 | 25 |
| 5.542 | 15.98 | 100 |
| 5.448 | 16.26 | 79 |
| 5.377 | 16.47 | 57 |
| 5.193 | 17.06 | 7 |
| 4.945 | 17.92 | 13 |
| 4.911 | 18.05 | 11 |
| 4.689 | 18.91 | 45 |
| 4.438 | 19.99 | 55 |
| 4.371 | 20.30 | 30 |
| 4.250 | 20.89 | 17 |
| 4.112 | 21.60 | 59 |
| 4.072 | 21.81 | 21 |
| 3.961 | 22.43 | 22 |
| 3.887 | 22.86 | 16 |
| 3.791 | 23.45 | 55 |
| 3.654 | 24.34 | 57 |
| 3.631 | 24.50 | 64 |
| 3.552 | 25.05 | 20 |
| 3.456 | 25.76 | 17 |
| 3.400 | 26.19 | 12 |
| 3.278 | 27.18 | 13 |
| 3.179 | 28.04 | 24 |
| 3.139 | 28.41 | 17 |
| 3.122 | 28.57 | 15 |
| 3.036 | 29.40 | 26 |
| 3.005 | 29.71 | 16 |
| 2.963 | 30.14 | 14 |
| 2.893 | 30.88 | 8 |
| 2.814 | 31.78 | 17 |
| 2.775 | 32.24 | 5 |
| 2.685 | 33.35 | 16 |
| 2.610 | 34.34 | 7 |
| 2.578 | 34.77 | 11 |
| 2.470 | 36.34 | 3 |
| 2.426 | 37.02 | 4 |
| 2.329 | 38.62 | 9 |

Optionally, crystalline polymorph C is additionally characterized by an infrared spectrum with bands observed at 3111, 2924, 2652, 2325, 2165, 2114, 2051, 1981, 1874, 1745, 1698, 1641, 1608, 1541, 1513, 1464, 1443, 1416, 1392, 1363, 1332, 1301, 1265, 1249, 1218, 1179, 1163, 1113, 1096, 1048, 1028, 995, 951, 926, 905, 823, 812, 774, 739, 713 cm⁻¹, as depicted in FIG. 4. Form C may thus provide an infrared spectrum substantially in accordance with FIG. 4.

The present invention also encompasses the Phosphate existing in non-solvated forms such as the polymorphic forms B, B1 or E. Such forms may be anhydrous, i.e. may be anhydrates, which may contain less than 2% by weight water, e.g. up to 1.5%, such as Forms B and B₁, or e.g. up to 0.5%, such as up to 0.2%, e.g. less than 0.1% by weight water, such as Form E. The presence of the above mentioned traces of water in polymorphic forms B, B₁ or E depend on the presence of humidity, which means that a high relative humidity, e.g. of about 80%, or higher, leads to a higher water content, and a low relative humidity, of e.g. up to 30%, to a lower water content of the above mentioned polymorphs. Preferably, the anhydrates mentioned above are essentially free of residual organic solvent media.

Thus, in a further aspect, the present invention provides a crystalline polymorphic form of 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, herein designated as Form B, characterised by an X-ray powder diffraction (XRPD) pattern having intensity peaks at values expressed in 2-theta degrees of about 4.19, 16.45, 17.01, 18.89 and 21.35. Crystalline polymorphic Form B may further present intensity peaks at any one or more values selected from the following values expressed in 2-theta degrees: about 8.44, 19.50, 20.86, 22.15, 25.67, 26.22 and 27.70.

In another aspect, crystalline polymorph B is characterised by an X-ray powder diffraction (XRPD) pattern substantially in accordance with Table 3 and FIG. 5.

Form B exists in an anhydrous form, e.g. containing up to 1.5% by weight water.

TABLE 3
X-Ray Powder Diffraction (XRPD) pattern of Form B showing
interplanar spacings
(d, given in Å, i.e. Angstroem), characteristic XRPD angles
(2 theta°) and relative intensities (in %)

| d-value | Angle | Rel. Intensity |
|---|---|---|
| (Å) | 2 theta° | (%) |
| 21.07 | 4.19 | 25 |
| 10.47 | 8.44 | 18 |
| 9.656 | 9.15 | 1 |
| 8.563 | 10.32 | 12 |
| 7.513 | 11.77 | 8 |
| 6.616 | 13.37 | 8 |
| 5.383 | 16.45 | 47 |
| 5.209 | 17.01 | 84 |
| 5.063 | 17.50 | 5 |
| 4.803 | 18.46 | 8 |
| 4.695 | 18.89 | 49 |
| 4.548 | 19.50 | 19 |
| 4.485 | 19.78 | 15 |
| 4.457 | 19.90 | 15 |
| 4.390 | 20.21 | 4 |
| 4.255 | 20.86 | 24 |
| 4.158 | 21.35 | 100 |
| 4.009 | 22.15 | 20 |
| 3.950 | 22.49 | 4 |
| 3.866 | 22.99 | 11 |
| 3.811 | 23.32 | 12 |
| 3.762 | 23.63 | 14 |
| 3.733 | 23.82 | 11 |
| 3.618 | 24.59 | 12 |
| 3.574 | 24.90 | 14 |
| 3.497 | 25.45 | 9 |
| 3.468 | 25.67 | 22 |
| 3.396 | 26.22 | 20 |
| 3.301 | 26.99 | 15 |
| 3.218 | 27.70 | 21 |
| 3.153 | 28.29 | 5 |
| 3.103 | 28.75 | 12 |
| 3.052 | 29.24 | 4 |
| 2.972 | 30.04 | 16 |
| 2.883 | 31.00 | 6 |
| 2.863 | 31.21 | 16 |
| 2.836 | 31.52 | 3 |
| 2.778 | 32.20 | 2 |
| 2.742 | 32.63 | 16 |
| 2.707 | 33.06 | 3 |
| 2.604 | 34.42 | 12 |
| 2.578 | 34.76 | 6 |
| 2.562 | 35.00 | 3 |
| 2.475 | 36.27 | 16 |
| 2.422 | 37.10 | 1 |
| 2.402 | 37.41 | 2 |
| 2.357 | 38.15 | 8 |
| 2.343 | 38.39 | 10 |
| 2.297 | 39.19 | 2 |
| 2.259 | 39.87 | 10 |

Optionally, crystalline polymorph B is additionally characterized by an infrared spectrum with bands observed at 3050, 2875, 2455, 2325, 2165, 2141, 2114, 2051, 1982, 1874, 1750, 1697, 1640, 1611, 1546, 1513, 1464, 1441, 1416, 13931366, 1333, 1318, 1301, 1284, 1244, 1219, 1181, 1161, 1114, 1097, 1081, 1044, 1030, 994, 948, 924, 896, 826, 812, 772, 741, 712 cm⁻¹, as depicted in FIG. 6. Form B may thus provide an infrared spectrum substantially in accordance with FIG. 6.

In an additional aspect, the present invention provides a crystalline polymorphic form of 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, herein designated as Form B1, which is characterised by an X-ray powder diffraction (XRPD) pattern having intensity peaks at values expressed in 2-theta degrees of about 16.46, 19.51, 19.76, 19.88 and 23.31. Crystalline polymorphic Form B1 may further present intensity peaks at any one or more values selected from the following values expressed in 2-theta degrees: about 8.39, 21.36, 23.00, 23.61, 23.80, 24.54, 26.20 and 27.71.

Form B1 exists in an anhydrous form, e.g. containing up to 1.5% by weight water.

In another aspect, crystalline polymorph B1 is characterised by an X-ray powder diffraction (XRPD) pattern substantially in accordance with Table 4 and FIG. 7.

TABLE 4
X-Ray Powder Diffraction (XRPD) pattern of Form B1 showing
interplanar spacings
(d, given in Å, i.e. Angstroem), characteristic XRPD angles
(2 theta°) and relative intensities (in %)

| d-value | Angle | Rel. Intensity |
|---|---|---|
| (Å) | 2 theta° | (%) |
| 21.06 | 4.19 | 14 |
| 10.54 | 8.39 | 24 |
| 9.652 | 9.16 | 2 |
| 8.585 | 10.30 | 20 |
| 7.509 | 11.78 | 10 |
| 6.613 | 13.38 | 8 |
| 5.381 | 16.46 | 100 |
| *5.254 | 16.86 | 17 |
| 5.197 | 17.05 | 19 |
| 5.061 | 17.51 | 10 |
| 4.809 | 18.43 | 10 |
| 4.692 | 18.90 | 21 |
| 4.546 | 19.51 | 31 |
| 4.489 | 19.76 | 40 |
| 4.452 | 19.88 | 41 |
| 4.388 | 20.22 | 9 |
| 4.266 | 20.81 | 13 |
| *4.156 | 21.36 | 24 |
| *4.127 | 22.52 | 17 |
| *4.008 | 22.16 | 21 |
| 3.953 | 22.47 | 6 |
| 3.864 | 23.00 | 27 |
| 3.814 | 23.31 | 29 |
| 3.765 | 23.61 | 29 |
| 3.736 | 23.80 | 29 |
| 3.625 | 24.54 | 24 |
| 3.581 | 24.85 | 15 |
| 3.504 | 25.40 | 13 |
| 3.466 | 25.68 | 6 |
| 3.398 | 26.20 | 27 |
| 3.299 | 27.00 | 11 |
| 3.216 | 27.71 | 24 |
| 3.158 | 28.24 | 11 |
| 3.102 | 28.76 | 14 |
| 3.050 | 29.25 | 4 |
| 2.971 | 30.06 | 6 |
| *2.944 | 30.33 | 5 |
| *2.890 | 30.92 | 5 |
| *2.870 | 31.13 | 7 |
| *2.843 | 31.44 | 4 |
| *2.790 | 32.06 | 4 |
| 2.743 | 32.61 | 12 |
| 2.709 | 33.05 | 4 |
| *2.689 | 33.29 | 4 |
| *2.658 | 33.69 | 1 |
| 2.605 | 34.40 | 7 |
| 2.578 | 34.77 | 6 |
| 2.563 | 34.99 | 6 |
| *2.499 | 35.91 | 4 |
| 2.476 | 36.25 | 10 |
| *2.405 | 37.36 | 2 |
| 2.356 | 38.16 | 6 |
| 2.344 | 38.38 | 5 |
| 2.297 | 39.18 | 4 |
| 2.258 | 39.89 | 2 |

Optionally, crystalline polymorph B1 is additionally characterized by an infrared spectrum being substantially the same as herein described for Form B, i.e. with bands observed at 3050, 2875, 2455, 2325, 2165, 2141, 2114, 2051, 1982, 1874, 1750, 1697, 1640, 1611, 1546, 1513, 1464, 1441, 1416, 13931366, 1333, 1318, 1301, 1284, 1244, 1219, 1181, 1161, 1114, 1097, 1081, 1044, 1030, 994, 948, 924, 896, 826, 812, 772, 741, 712 cm⁻¹, as depicted in FIG. 6. Form B1 may thus provide an infrared spectrum substantially in accordance with FIG. 6.

Form B1 differs from Form B with respect to the X-ray powder diffraction (XRPD) pattern only with regard to relative intensities of the pattern, whereas d-values are within the given measurement accuracy of 0.05 degrees/2 theta. Some reflections of Form B1 have a better resolution resulting in additional reflections which are marked with an asterisk in Table 4.

Thus Form B and Form B1 have the same infrared spectrum, but differ with respect to their X-ray powder diffraction (XRPD) patterns, as well as with regard to certain properties such as humidity sorption properties and to their different morphology, such as their crystal sizes as seen in electronic microscopy. Furthermore, Form B and B1 may occur as mixtures.

Form B and B1 have a melting point in the range of 175-176° C. (Kofler).

In a further aspect, the present invention provides a crystalline polymorphic form of 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, herein designated as Form E, which is characterised by an X-ray powder diffraction (XRPD) pattern having intensity peaks at values expressed in 2-theta degrees of about 4.60, 13.39, 18.20, 18.53 and 22.75. Crystalline polymorphic Form E may further present intensity peaks at any one or more values selected from the following values expressed in 2-theta degrees: about 22.20, 22.99, 23.24, 24.19 and 30.50.

Form E exists in an anhydrous form, e.g. containing up to 0.5% by weight water.

In another aspect, crystalline polymorph E characterised by an X-ray powder diffraction (XRPD) pattern substantially in accordance with Table 5 and FIG. 8.

TABLE 5
X-Ray Powder Diffraction (XRPD) pattern of Form E showing
interplanar spacings
(d, given in Å, i.e. Angstroem), characteristic XRPD angles
(2 theta°) and relative intensities (in %)

| d-value | Angle | Rel. Intensity |
|---|---|---|
| (Å) | 2 theta° | (%) |
| 19.19 | 4.60 | 44 |
| 10.92 | 8.09 | 3 |
| 9.570 | 9.23 | 2 |
| 6.609 | 13.39 | 43 |
| 6.332 | 13.97 | 5 |
| 6.263 | 14.13 | 5 |
| 5.964 | 14.84 | 5 |
| 5.749 | 15.40 | 9 |
| 5.450 | 16.25 | 2 |
| 5.152 | 17.20 | 7 |
| 5.075 | 17.46 | 5 |
| 4.870 | 18.20 | 27 |
| 4.785 | 18.53 | 100 |
| 4.591 | 19.32 | 3 |
| 4.382 | 20.25 | 3 |
| 4.294 | 20.67 | 7 |
| 4.170 | 21.29 | 4 |
| 4.123 | 21.54 | 5 |
| 4.001 | 22.20 | 12 |
| 3.906 | 22.75 | 49 |
| 3.865 | 22.99 | 20 |
| 3.824 | 23.24 | 13 |
| 3.799 | 23.40 | 7 |
| 3.757 | 23.66 | 9 |
| 3.677 | 24.19 | 23 |
| 3.626 | 24.53 | 2 |
| 3.606 | 24.67 | 2 |
| 3.545 | 25.10 | 2 |
| 3.500 | 25.43 | 3 |
| 3.477 | 25.60 | 3 |
| 3.400 | 26.19 | 3 |
| 3.338 | 26.68 | 6 |
| 3.308 | 26.93 | 1 |
| 3.206 | 27.80 | 2 |
| 3.189 | 27.96 | 2 |
| 3.108 | 28.70 | 1 |
| 3.097 | 28.81 | 2 |
| 3.061 | 29.15 | 1 |
| 3.011 | 29.65 | 4 |
| 3.000 | 29.75 | 4 |
| 2.929 | 30.50 | 10 |
| 2.861 | 31.24 | 4 |
| 2.817 | 31.74 | 3 |
| 2.783 | 32.14 | 2 |
| 2.729 | 32.79 | 2 |
| 2.695 | 33.21 | 4 |
| 2.658 | 33.69 | 4 |
| 2.641 | 33.91 | 5 |
| 2.586 | 34.65 | 1 |
| 2.523 | 35.55 | 2 |
| 2.493 | 36.00 | 1 |
| 2.477 | 36.24 | 1 |
| 2.449 | 36.67 | 2 |
| 2.388 | 37.63 | 5 |
| 2.342 | 38.41 | 2 |

Optionally, crystalline polymorph E is additionally characterized by an infrared spectrum with bands observed at 2918, 2702, 2417, 2324, 2165, 2051, 1982, 1752, 1700, 1642, 1610, 1546, 1512, 1468, 1443, 1419, 1395, 1364, 1331, 1303, 1238, 1181, 1165, 1140, 1096, 1052, 1029, 1008, 953, 906, 882, 831, 819, 768, 739, 714, 663 cm⁻¹, as depicted in FIG. 9. Form E may thus provide an infrared spectrum substantially in accordance with FIG. 9.

Form E has a melting point in the range of 167-172° C. (Kofler).

Depending on the solvent from which the Phosphate is recovered, the Phosphate may be obtained as a solvate other than a hydrate such as polymorphic Form D. Such solvates form part of the present invention, and references to the Phosphate hereinafter include solvates thereof.

Thus, in a further aspect, the present invention provides a crystalline polymorphic form of 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to the phosphate is 1:1, herein designated as Form D, which is characterised by an X-ray powder diffraction (XRPD) pattern having intensity peaks at values expressed in 2-theta degrees of about 14.33, 16.05, 16.36, 21.97 and 22.89. Crystalline polymorphic Form D may further present intensity peaks at any one or more values selected from the following values expressed in 2-theta degrees: about 4.75, 15.04, 16.70, 19.26, 19.57, 20.80, 21.97, 22.74, 23.91 and 24.53.

Form D is in the form of a solvate with methanol.

In another aspect, crystalline polymorph D characterised by an X-ray powder diffraction (XRPD) pattern substantially in accordance with Table 6 and FIG. 10.

TABLE 6
X-Ray Powder Diffraction (XRPD) pattern of Form D showing
interplanar spacings
(d, given in Å, i.e. Angstroem), characteristic XRPD angles
(2 theta°) and relative intensities (in %)

| d-value | Angle | Rel. Intensity |
|---|---|---|
| (Å) | 2 theta° | (%) |
| 20.76 | 4.25 | 24 |
| 18.61 | 4.75 | 33 |
| 12.36 | 7.15 | 9 |
| 10.70 | 8.26 | 12 |
| 10.31 | 8.57 | 15 |
| 9.960 | 8.87 | 5 |
| 9.193 | 9.61 | 8 |
| 8.538 | 10.35 | 20 |
| 7.176 | 12.33 | 9 |
| 6.852 | 12.91 | 15 |
| 6.177 | 14.33 | 100 |
| 5.887 | 15.04 | 39 |
| 5.517 | 16.05 | 40 |
| 5.414 | 16.36 | 63 |
| 5.304 | 16.70 | 35 |
| 5.135 | 17.26 | 26 |
| 4.926 | 17.99 | 24 |
| 4.748 | 18.67 | 8 |
| 4.605 | 19.26 | 31 |
| 4.534 | 19.57 | 36 |
| 4.396 | 20.18 | 21 |
| 4.312 | 20.58 | 24 |
| 4.268 | 20.80 | 32 |
| 4.094 | 21.69 | 26 |
| 4.043 | 21.97 | 41 |
| 3.977 | 22.34 | 25 |
| 3.907 | 22.74 | 36 |
| 3.882 | 22.89 | 43 |
| 3.786 | 23.48 | 18 |
| 3.719 | 23.91 | 35 |
| 3.672 | 24.22 | 42 |
| 3.627 | 24.53 | 37 |
| 3.539 | 25.14 | 20 |
| 3.448 | 25.82 | 28 |
| 3.412 | 26.10 | 27 |
| 3.279 | 27.18 | 16 |
| 3.246 | 27.45 | 12 |
| 3.204 | 27.82 | 8 |
| 3.113 | 28.66 | 8 |
| 3.036 | 29.39 | 10 |
| 3.002 | 29.73 | 8 |
| 2.949 | 30.29 | 11 |
| 2.931 | 30.47 | 12 |
| 2.905 | 30.75 | 18 |
| 2.855 | 31.31 | 4 |
| 2.730 | 32.78 | 13 |
| 2.664 | 33.62 | 7 |
| 2.629 | 34.08 | 11 |
| 2.564 | 34.97 | 4 |
| 2.514 | 35.68 | 4 |
| 2.487 | 36.08 | 6 |
| 2.473 | 36.30 | 5 |
| 2.449 | 36.67 | 3 |
| 2.378 | 37.81 | 7 |
| 2.307 | 39.01 | 11 |
| 2.724 | 32.85 | 2 |
| 2.615 | 34.27 | 4 |
| 2.570 | 34.88 | 3 |
| 2.555 | 35.10 | 3 |
| 2.453 | 36.61 | 2 |
| 2.419 | 37.13 | 2 |
| 2.334 | 38.55 | 2 |
| 2.305 | 39.04 | 3 |
| 2.276 | 39.57 | 3 |

Optionally, crystalline polymorph D is additionally characterized by an infrared spectrum with bands observed at 3129, 2933, 2684, 2325, 2165, 2150, 2113, 2051, 1982, 1743, 1699, 1641, 1604, 1538, 1511, 1467, 1446, 1412, 1389, 1357, 1332, 1303, 1279, 1242, 1164, 1107, 1077, 1063, 1021, 994, 956, 928, 903, 832, 802, 769, 739, 719 cm⁻¹, as depicted in FIG. 11. Form D may thus provide an infrared spectrum substantially in accordance with FIG. 11.

### BRIEF DESCRIPTION OF THE DRAWINGS

FIG. 1 shows the X-ray powder diffraction (XRPD) pattern of Form A

FIG. 2 shows the infrared spectrum of Form A

FIG. 3 shows the X-ray powder diffraction (XRPD) pattern of Form C

FIG. 4 shows the infrared spectrum of Form C

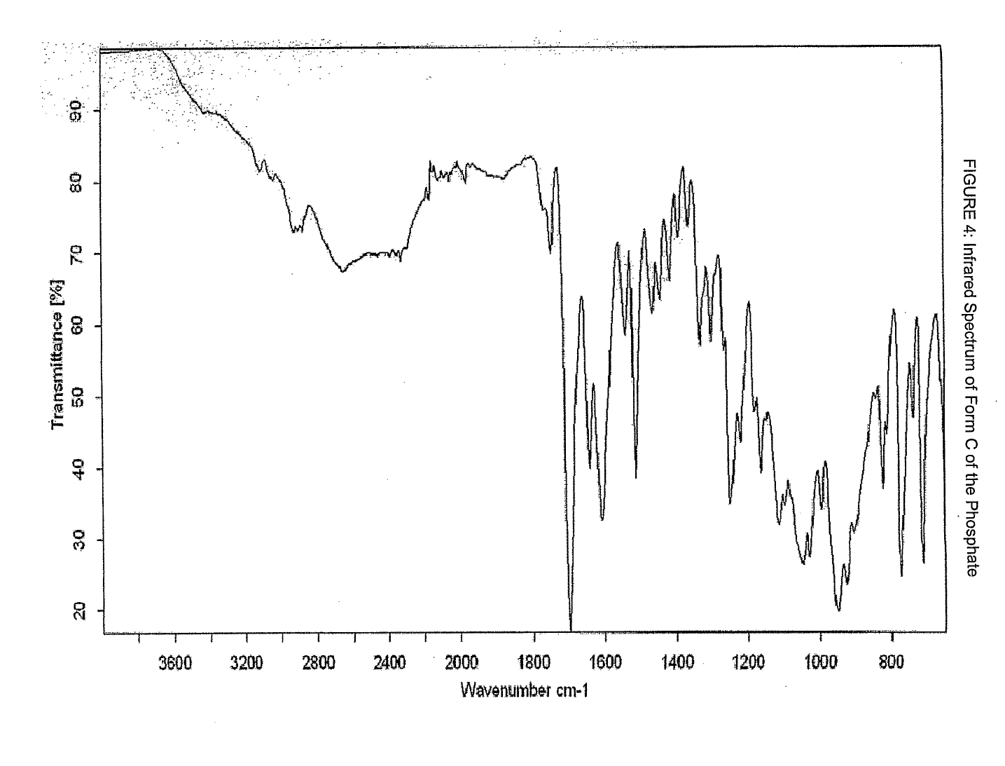

FIG. 5 shows the X-ray powder diffraction (XRPD) pattern of Form B

FIG. 6 shows the infrared spectrum of Form B and Form B1

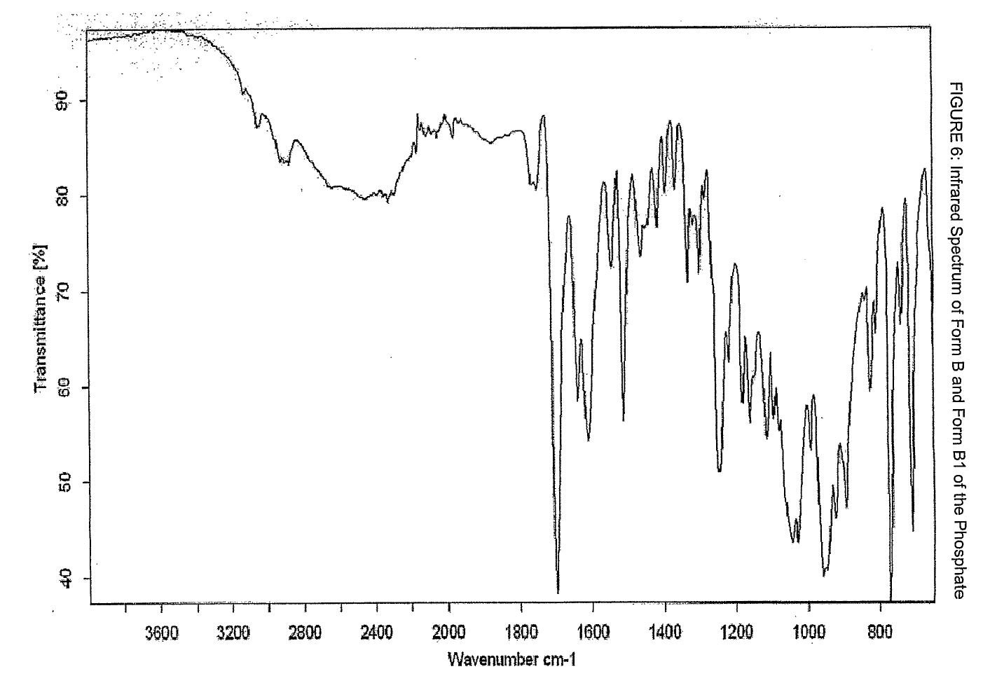

FIG. 7 shows the X-ray powder diffraction (XRPD) pattern of Form B1

FIG. 8 shows the X-ray powder diffraction (XRPD) pattern of Form E

FIG. 9 shows the infrared spectrum of Form E

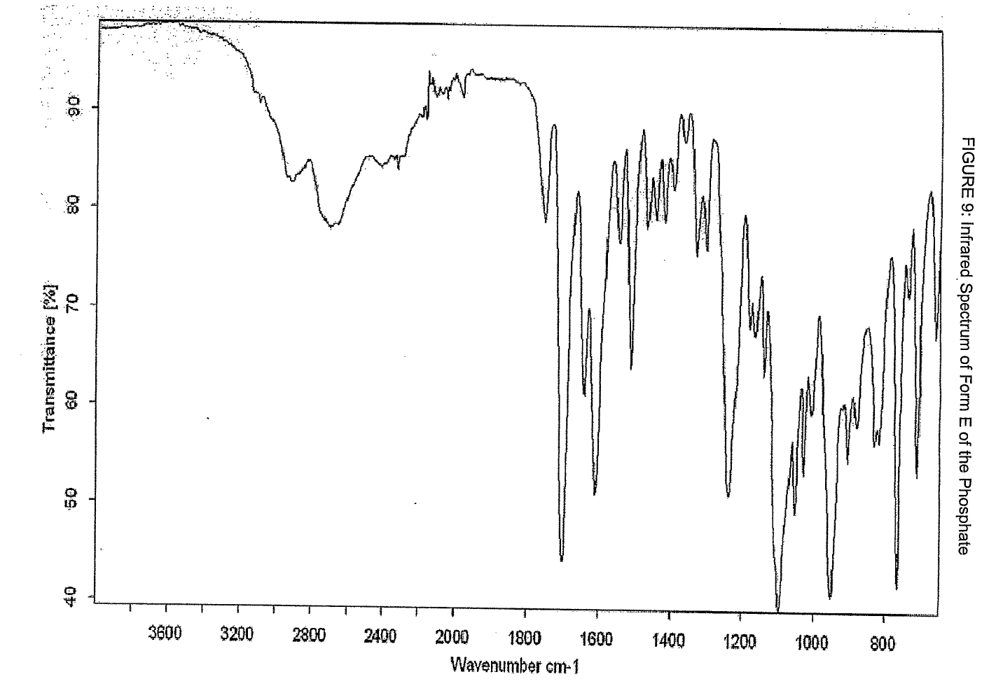

FIG. 10 shows the X-ray powder diffraction (XRPD) pattern of Form D

FIG. 11 shows the infrared spectrum of Form D

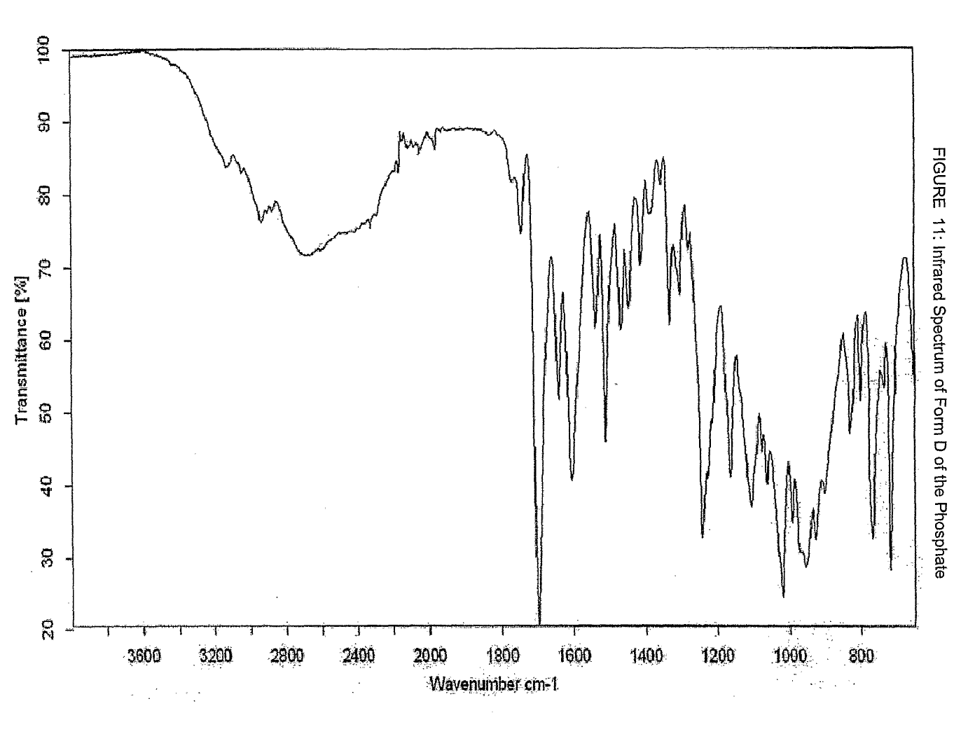

In all Figures showing the infrared spectrum of a polymorphic form of the Phosphate, the scale of the abscissa is the wave number in cm⁻¹, and the ordinate is transmittance in %. In all Figures showing the X-ray powder diffraction (XRPD) pattern of a polymorphic form of the Phosphate, the scale of the abscissa is in degrees 2θ (2-theta scale), and the ordinate is the linear intensity in counts per second (cps)

The present invention encompasses the Phosphate, and its crystalline polymorphic forms A, B, B1, C, D and E, when isolated in pure form or as mixtures of said polymorphs, or when admixed with other materials, e.g. pharmaceutically acceptable carriers.

Thus in one aspect there is provided the 5-[[4-[2-(methyl-2-pyridinylamino)ethoxy]phenyl]methyl]-2,4-thiazolidinedione phosphate, and its polymorphic forms A, B, B1, C, D and E, in isolated form.

In a further aspect there is provided the 5-[[4-[2-(methyl-2-pyridinylamino)ethoxy]phenyl]methyl]-2,4-thiazolidinedione phosphate, and its polymorphic forms A, B, B1, C, D and E, in substantially pure form.

In another aspect of the invention there is provided the Phosphate, and its polymorphic forms A, B, B1, C, D and E as mixtures thereof.

The Phosphate, preferably as the Phosphate Hydrate or the Phosphate Anhydrate, also exists in non-crystalline form, i.e. amorphous form, which may be prepared according, e.g. analogous to, conventional methods, e.g. by preparing a solution of 5-[[4-[2-(methyl-2-pyridinylamino) ethoxy]phenyl]methyl]-2,4-thiazolidinedione phosphate in a mixture comprising a ketone, e.g. acetone, or in a alcohol, e.g. ethanol, and water, and spray-drying said solution. Alternatively, quick precipitation may be performed according, e.g. analogous to known methods.

The present invention also encompasses the Phosphate, and its polymorphic forms A, B, B1, C, D and E, in e.g. bulk form, such form being capable of being further processed, e.g. milled, according, e.g. analogous to known processes. The invention further encompasses the Phosphate, and its polymorphic forms A, B, B1, C and E, in a pharmaceutically acceptable form, e.g. in a milled form.

Furthermore the present invention is directed to processes for the preparation of the Phosphate and its polymorphic forms A, B, B1, C, D and E.

Thus, the present invention provides a process for preparing the 5-[[4-[2-(methyl-2-pyridinylamino)ethoxy]phenyl]methyl]-2,4-thiazolidinedione phosphate comprising reacting 5-[[4-[2-(methyl-2-pyridinylamino)ethoxy]phenyl]methyl]-2,4-thiazolidinedione or a salt thereof, dispersed or suspended or dissolved in a suitable solvent medium, with a suitable source of a phosphate ion.

Optionally, thereafter a solvate of the 5-[[4-[2-(methyl-2-pyridinylamino)ethoxy]phenyl]methyl]-2,4-thiazolidinedione phosphate may be formed as described below, e.g. within the reaction mixture obtained by admixing 5-[[4-[2-(methyl-2-pyridinylamino)ethoxy]phenyl]methyl]-2,4-thiazolidinedione or a salt thereof, dispersed or suspended or dissolved in a suitable solvent medium, and the suitable source of a phosphate ion as described above.

Optionally, 5-[[4-[2-(methyl-2-pyridinylamino)ethoxy]phenyl]methyl]-2,4-thiazolidinedione phosphate, preferably in one of its polymorphic forms, may be recovered from the reaction mixture as described below.

Optionally, said 5-[[4-[2-(methyl-2-pyridinylamino)ethoxy]phenyl]methyl]-2,4-thiazolidinedione phosphate, preferably in one of its polymorphic forms, may be dried, preferably under vacuum.

Optionally, one polymorphic form may be converted into another one according, e.g. analogously to known methods. For example, Form A may be converted to Form B or D, Form C may be converted to Form B or B1, Form D may be converted to Form A or B, Forms A, B, B1, D and E may be converted to Form C, under the conditions and/or according to the processes described below.

Alternatively, the conversion of one polymorphic form into another one may take place in the reaction mixture obtained by contacting 5-[[4-[2-(methyl-2-pyridinylamino)ethoxy]phenyl]methyl]-2,4-thiazolidinedione in a suitable solvent medium with a suitable source of phosphate ion as herein described.

5-[[4-[2-(methyl-2-pyridinylamino)ethoxy]phenyl]methyl]-2,4-thiazolidinedione phosphate, preferably in one of its polymorphic forms, may furthermore be processed according to known manufacturing processes, e.g. may be milled.

In another aspect, the invention provides a process for preparing the 5-[[4-[2-(methyl-2-pyridinylamino)ethoxy]phenyl]methyl]-2,4-thiazolidinedione phosphate, in its polymorphic forms A, B, B1 or E, comprising reacting 5-[[4-[2-(methyl-2-pyridinylamino)ethoxy]phenyl]methyl]-2,4-thiazolidinedione or a salt thereof, dispersed or suspended or dissolved in a suitable solvent medium, with a suitable source of a phosphate ion, and thereafter, carrying out the following steps:
(i) optionally forming a solvate of the 5-[[4-[2-(methyl-2-pyridinylamino)ethoxy]phenyl]methyl]-2,4-thiazolidinedione phosphate,
(ii) recovering the 5-[[4-[2-(methyl-2-pyridinylamino)ethoxy]phenyl]methyl]-2,4-thiazolidinedione phosphate,
(iii) drying the 5-[[4-[2-(methyl-2-pyridinylamino)ethoxy]phenyl]methyl]-2,4-thiazolidinedione phosphate obtained in step ii), especially under vacuum, to obtain the 5-[[4-[2-(methyl-2-pyridinylamino)ethoxy]phenyl]methyl]-2,4-thiazolidinedione phosphate in its polymorphic form A, B, B1 or E.

Optionally, the Phosphate, and its polymorphic forms A, B, B1 or E, as obtained by the above described process may be further processed in known manufacturing processes, e.g. in a milling process.

Preferably, the suitable source of the phosphate ion in the above mentioned processes is phosphoric acid.

Alternatively, 5-[[4-[2-(methyl-2-pyridinylamino)ethoxy]phenyl]methyl]-2,4-thiazolidinedione or a salt thereof, may be added as a powder to the suitable source of the phosphate ion.

In general Phosphates and its polymorphic forms A, B, B1 or E may be prepared by contacting stoichiometric amounts, for example 1:1, of phosphoric acid and 5-[[4-[2-(methyl-2-pyridinylamino)ethoxy]phenyl]methyl]-2,4-thiazolidinedione, or alternatively using an excess of phosphoric acid, e.g. a ratio of 1.1:1, or 2:1 to 2.5:1 of phosphoric acid and 5-[[4-[2-(methyl-2-pyridinylamino)ethoxy]phenyl]methyl]-2,4-thiazolidinedione.

The concentration of 5-[[4-[2-(methyl-2-pyridinylamino)ethoxy]phenyl]methyl]-2,4-thiazolidinedione is preferably in the range of 1 to 50% weight/volume, more preferably 1-10% weight/volume related to the total amount of solvent medium used in the reaction.

A suitable solvent medium for the solution or dispersion or suspension of 5-[[4-[2-(methyl-2-pyridinylamino)ethoxy]phenyl]methyl]-2,4-thiazolidinedione or a salt thereof, and for the reaction with a suitable source of the phosphate ion, as described above, is an organic solvent medium, e.g. a ketone, e.g. acetone, or an alcohol, e.g. a C₁-C₄ alcohol, e.g. ethanol or methanol, or a nitrile, e.g. acetonitrile, or an ether, e.g. tetrahydrofuran, or mixtures thereof, or water, or mixtures of said organic solvent media with water.

Preferably, water is used as a cosolvens. Preferred amounts of water are 1 to 100% (v/v), preferably 1 to 20% (v/v) of water related to the organic solvent medium.

A suitable source of the phosphate ion is phosphoric acid, for example 85% (w/w) phosphoric acid or less concentrated phosphoric acid, e.g. diluted from 1:1 to 1:10 w/v with water or with an organic solvent medium such as a ketone, e.g. acetone, or an alcohol, e.g. a C₁-C₄ alcohol, e.g. ethanol or methanol, or mixtures of a ketone and an alcohol. The phosphoric acid is preferably added as such, or as a solution, for example a solution in one of the above mentioned organic solvent media.

An alternative source of the phosphate ion may be metaphosphoric acid, preferably in combination with water, or sodium or potassium dihydrogenphosphate, disodium or dipotassium hydrogenphosphate or trisodium or tripotassium phosphate in combination with a mineral acid, preferably phosphoric acid.

Formation of the Phosphate Hydrate, e.g. of Form A, requires the presence of water at some stage. The water may be present in the source of the phosphate ion, e.g. in the phosphoric acid used, e.g. by using 85% (w/w) or less concentrated phosphoric acid, or the water may be present as a cosolvens in the process, e.g. 1 to 100% (v/v), preferably 1-20%, of water related to the organic solvent medium.

However, it is also possible to provide sufficient water for the formation of the Phosphate Hydrate, such as Form A, by carrying out the reaction with exposure to atmospheric moisture, or by the use of a non-anhydrous solvent medium, e.g. aqueous acetone, or of a non-anhydrous source of the phosphate ion, e.g. 85% (w/w) phosphoric acid.

The reaction may be carried out at room temperature or at elevated temperatures of e.g. about 35° C. to about 60° C., preferably at about 30° C. to about 50° C., or at the reflux temperature of the solvent medium, although any convenient temperature that provides the required product may be employed.

Solvates, preferably the hydrates, of the Phosphate may be prepared, e.g. by crystallising from a solvent medium as described above which may provide or contain the solvate moiety, or by exposing the Phosphate to the solvate moiety as a vapour, according, e.g. analogously to known methods. The formation of such solvates may take place in the reaction mixture as described above.

Recovery of the required compound, e.g. the Phosphate, for example in its polymorphic forms, before drying comprises isolation from the reaction mixture and/or from an appropriate solvent medium, which is optionally the above mentioned solvent medium used for the above described reaction, preferably with water as a cosolvens, or which is a mixture of said solvent media, or alternatively a different solvent medium or mixture thereof, e.g. a C₁-C₄alkyl acetate, or e.g. a hydrogenated carbon, e.g. hexane. The isolation of the required compound from the reaction mixture and/or solvent medium as described above may be performed by filtration according to known methods, and may further comprise a subsequent washing step which means that the required compound may be washed in one of the solvent media described above, e.g. in ethanol, such as 96% (w/w) ethanol, or in mixtures thereof, e.g. in a mixture of acetone and water, e.g. in a 95% (v/v) mixture of acetone and water.

Alternatively the required compound may be isolated by crystallisation from the reaction mixture and/or from the appropriate solvent medium or mixture of solvent media as described above which may be initiated by the use of seed crystals. Careful control of precipitation temperature from approximately 20° C. to 80° C. to about 0° C. to 20° C., and/or the use of seed crystals are useful to improve the reproducibility of the Phosphate, and its polymorphic forms, such as Forms A, B, B1 and E.

Preferably the isolated Phosphate, for example in its polymorphic forms A, B, B1 and E, is dried under vacuum at room temperature, e.g. at a temperature of about 20° C. to about 35° C., e.g. at about 25° C., or at elevated temperatures, e.g. at about 35° C. to about 80° C., such as about 40° C. to about 60° C., preferably at about 40° C. The drying is optionally carried out using a desiccant, e.g. phosphorus pentoxide. Drying is continued until the water content is below approximately 4.5%, e.g. 3.58%, e.g. less than 0.1% by weight. The duration of the drying procedure is not critical and may be for instance about 10 to 30 hours, e.g. 15 to 25 hours, preferably about 18 to 20 hours.

In a preferred embodiment, Form A may be prepared by reacting 5-[[4-[2-(methyl-2-pyridinylamino)ethoxy]phenyl]methyl]-2,4-thiazolidinedione or a salt thereof, dispersed or suspended or dissolved in a suitable solvent medium, e.g. in a mixture of acteone and water, with a suitable source of a phosphate ion, e.g. 85% phosphoric acid. Optionally seed crystals of Form A may be added, and the mixture obtained may be stirred e.g. for about 3 to 5 hours at a temperature as described above, e.g. at about room temperature. Subsequently, 5-[[4-[2-(methyl-2-pyridinylamino)ethoxy]phenyl]methyl]-2,4-thiazolidinedione phosphate in its polymorphic form A may be isolated from the mixture as described above, e.g. by filtration, and may be washed with a suitable solvent medium, e.g. with a mixture of acetone and water, and may subsequently be dried at a temperature described above, preferably at about room temperature and under vacuum.

In a further preferred embodiment, Form B may be prepared by reacting 5-[[4-[2-(methyl-2-pyridinylamino)ethoxy]phenyl]methyl]-2,4-thiazolidinedione or a salt thereof, dispersed or suspended or dissolved in a suitable solvent medium, e.g. in a mixture of acetone and water, with a suitable source of a phosphate ion, e.g. 85% phosphoric acid. Optionally seed crystals of Form B may be added, and the mixture obtained may be stirred for at least about 30 hours at a temperature as described above, e.g. at about room temperature. Subsequently, 5-[[4-[2-(methyl-2-pyridinylamino)ethoxy]phenyl]methyl]-2,4-thiazolidinedione phosphate in its polymorphic form C may be isolated from the mixture as described above, e.g. by filtration, and may be washed with a suitable solvent medium, e.g. with a mixture of acetone and water, and may subsequently be dried at a temperature described above, preferably at a temperature of about 40° C., and under vacuum, to obtain Form B, which may contain traces of B1.

In another preferred embodiment, Form B1 may be prepared according to a process similar to the process described above for B, but optionally using seed crystals of Form B1 instead of those of Form B, and stirring the mixture for at least about 50 hours.

In a further preferred embodiment, Form E may be prepared by reacting 5-[[4-[2-(methyl-2-pyridinylamino)ethoxy]phenyl]methyl]-2,4-thiazolidinedione or a salt thereof, dispersed or suspended or dissolved in a suitable solvent medium, e.g. in ethanol, such as 96% (w/w) ethanol, with a suitable source of a phosphate ion, e.g. 85% phosphoric acid, at an elevated temperature as described above. The reaction mixture obtained may subsequently be cooled to about room temperature under stirring. Subsequently, 5-[[4-[2-(methyl-2-pyridinylamino) ethoxy]phenyl]methyl]-2,4-thiazolidinedione phosphate in its polymorphic form E may be isolated from the reaction mixture as described above, e.g. by filtration, and may be washed with a suitable solvent medium, e.g. with ethanol, such as 96% (w/w) ethanol, and may subsequently be dried at a temperature described above, preferably at a temperature of about 40° C., and under vacuum.

Form A may be converted to Form B by heating Form A to approximately 140° C. to about 160° C.

In another aspect, the present invention provides a process for preparing the Phosphate in its polymorphic Form C comprising the following steps:
(i) dispersing or suspending or dissolving 5-[[4-[2-(methyl-2-pyridinylamino) ethoxy]phenyl]methyl]-2,4-thiazolidinedione phosphate in its polymorphic forms A, B, B1, D or E, in a suitable solvent medium to obtain a mixture,
(ii) stirring the mixture obtained in step (i) alternately for about 1 hour at about 50° C. and subsequently for about 1 hour at about 10° C., for a total of about 3 to about 5 days,
(iii) recovering the product, i.e. polymorph C, from the mixture obtained in step (ii), and
(iv) air-drying the product obtained in step (iii).

The term “mixture” as used herein with regard to the processes for the preparation of polymorphic forms C and D is understood to include a dispersion, a suspension and/or a solution of a given compound, e.g. of 5-[[4-[2-(methyl-2-pyridinylamino)ethoxy]phenyl]methyl]-2,4-thiazolidinedione phosphate, e.g. in one of its polymorphic forms, in a suitable solvent medium.

Step (ii) of the above mentioned process may be carried out by stirring the mixture obtained in step (i) alternately for about 1 hour at a temperature of about 30° C. to 50° C., preferably at about 50° C., and subsequently for about 1 hour at about 0° C. to about 20° C., preferably at about 1° C., for a total of about 3 to about 5 days. Alternatively, the alternating stirring procedure may be interrupted overnight by keeping the mixture at room temperature and continuing the alternating stirring procedure on the subsequent day.

The preferred solvent medium of the above mentioned process to prepare Form C is a mixture of acetone and water, e.g. having a ratio of about 2:1 (v/v) of acetone to water. Step (iii) of said process may be performed by isolating the product, i.e. Form C, from the mixture by filtration and washing it with a mixture of acetone and water, e.g. having in a ratio of acetone to water of about 95:5 (v/v). Preferably, the isolation is performed from a mixture having a temperature of about 0° C. to about 30° C., preferably of about 10° C. Air-drying of step (iv) may be performed for about 5 hours to about 20 hours, e.g. for about 10 hours.

The term “air-drying” as used herein is understood to mean drying a compound, e.g. a polymorph of Form C, in the open air, with a relative humidity of about 20% to about 80%, e.g. of about 30% to about 60%, e.g. of about 40% to about 50%, and at a temperature of about 18° C. to about 25° C., e.g. at about 22° C.

Form C prepared by said process may be converted to Form B1 by performing the drying step (iv) at a temperature of about 40° C. or higher, e.g. of about 60° C. to about 80° C., preferably of about 50° C., optionally in vacuo, for about 5 hours to about 20 hours, e.g. for about 10 hours.

Alternatively, Form C may be prepared by a process comprising the following steps:
(i) dissolving or dispersing or suspending 5-[[4-[2-(methyl-2-pyridinylamino) ethoxy]phenyl]methyl]-2,4-thiazolidinedione phosphate of Form A, B, B1, D or E in a suitable solvent medium to obtain a mixture,
(ii) adding a suitable source of a phosphate ion, e.g. phosphoric acid, to the mixture obtained in step (i),
(iii) recovering the product, i.e. Form C, from the mixture obtained in step (ii), and
(iv) air-drying the product obtained in step (iii).

The preferred solvent medium of the above described alternative process for the preparation of Form C is a mixture of acetone and water, e.g. in a ratio of about 1:1 (v/v) of acetone to water. Step (iii) may be performed by isolating the product, i.e. Form C, by filtration and washing it with a mixture of acetone and water, e.g. having a ratio of acetone to water of about 95:5 (v/v). Air-drying of step (iv) may be performed for about 5 hours to about 20 hours, e.g. for about 10 hours.

Form C prepared according to the above described alternative process may be converted to Form B by drying Form C as obtained in step (iv) at a temperature of about 40° C. or higher, e.g. of about 60° C. to about 80° C., preferably of about 50° C., optionally in vacuo, for about 5 hours to about 20 hours, e.g. for about 10 hours.

In a further aspect, the present invention provides a process for preparing the Phosphate in its polymorphic Form D comprising the following steps:
(i) dissolving or dipersing or suspending 5-[[4-[2-(methyl-2-pyridinylamino)ethoxy]phenyl]methyl]-2,4-thiazolidinedione phosphate in its polymorphic Form A in a suitable solvent medium to obtain a mixture,
(ii) heating the mixture obtained in step (i) to a temperature of about 60° C. for about 4 hours, followed by cooling the mixture to about room temperature under stirring,
(iii) recovering the product, i.e. polymorph D, from the mixture obtained in step (ii), and
(iv) drying the product obtained in step (iii), preferably in vacuo.

Step (ii) may be performed by heating the mixture obtained in step (i) to a temperature of about 40° C. to about 60° C. for about 2 hours to about 6 hours, followed by cooling the mixture to about room temperature under stirring. Preferably, step (ii) is carried out a temperature of about 60° C. for about 4 hours.

The preferred suitable solvent medium used in the above mentioned process is methanol.

Step (iii) of said process may be performed by isolating the product, i.e. Form D, by filtration and washing it with methanol. The drying in step (iv) may be performed at a temperature of 20° C. to about 60° C., preferably of about 25° C. to about 30° C. for about 5 hours to about 20 hours, e.g. for about 10 hours.

Form D may contain residual solvent, e.g. methanol, and in this case is not suitable to be used in the pharmaceutical compositions mentioned below. Form D may, however, be converted to Form A upon exposure to humidity, e.g. at about 60% to about 70% relative humidity. Furthermore, Form D may loose its residual content of methanol upon heating to a temperature of not less than 60° C., or may be converted to Form B by heating to about 120° C. or higher. Both Forms A and B are then suitable for incorporation into pharmaceutical compositions.

Optionally, the required compound, e.g. the Phosphate, preferably in its polymorphic forms A, B, B1, C, D and E, may be further processed without being isolated from the mixture of the reaction as described above.

5-[[4-[2-(methyl-2-pyridinylamino)ethoxy]phenyl]methyl]-2,4-thiazolidinedione may be prepared according to known procedures, such as the method disclosed in EP-A-0306228.

As mentioned above the compound of the invention, i.e. the Phosphate and its polymorphic forms A, B, B1, C and E have useful therapeutic properties. The present invention accordingly provides 5-[[4-[2-(methyl-2-pyridinylamino)ethoxy]phenyl]methyl]-2,4-thiazolidinedione phosphate, and its polymorphic forms A, B, B1, C and E, or a mixture thereof, for use as a pharmaceutically active substance, e.g. for use as a medicament.

The term “the Phosphate, and its polymorphic forms A, B, B1, C and E” or “5-[[4-[2-(methyl-2-pyridinylamino)ethoxy]phenyl]methyl]-2,4-thiazolidinedione phosphate, and its polymorphic forms A, B, B1, C and E”, respectively, as herein used related to pharmaceutical and/or therapeutic use or compositions, is understood to mean these compounds either (used) as a single component or as a mixture thereof.

Particularly, the present invention provides the Phosphate, and its polymorphic forms A, B, B1, C and E, for use in the treatment and/or prophylaxis of hyperglycaemia in a human and non-human mammal. More particularly, the present invention provides the 5-[[4-[2-(methyl-2-pyridinylamino)ethoxy]phenyl]methyl]-2,4-thiazolidinedione phosphate, and its polymorphic forms A, B, B1, C and E, or a mixture thereof, for use in the treatment and/or prophylaxis of diabetes mellitus, conditions associated with diabetes mellitus and certain complications thereof in a human or non-human mammal.

When used herein, the term “prophylaxis of conditions associated with diabetes mellitus” includes treating conditions such as insulin resistance, impaired glucose tolerance, hyperinsulinaemia and gestational diabetes. Diabetes mellitus preferably means Type II diabetes mellitus. Conditions associated with diabetes mellitus include hyperglycaemia, hyperlipidaemia, obesity, hypertension, cardiovascular disease, certain eating disorders, polycystic ovarian syndrome and steroid induced insulin resistance. Complications of conditions associated with diabetes mellitus encompassed herein include renal disease, especially renal disease associated with the development of Type II diabetes mellitus including diabetic nephropathy, glomerulonephritis, glomerular sclerosis, nephrotic syndrome, hypertensive nephrosclerosis and end stage renal disease.

The Phosphate, and its polymorphic forms A, B, B1, C and E, may be administered per se, or preferably, as a pharmaceutical composition also comprising a pharmaceutically acceptable carrier.

Accordingly, the present invention also provides a pharmaceutical composition comprising 5-[[4-[2-(methyl-2-pyridinylamino)ethoxy]phenyl]methyl]-2,4-thiazolidinedione phosphate, or one of its polymorphic forms A, B, B1, C and E, or a mixture thereof, and a pharmaceutically acceptable carrier.

As used herein, the term “pharmaceutically acceptable” embraces compounds, compositions and ingredients for both human and veterinary use.

In accordance with conventional pharmaceutical practice the carrier may comprise a diluent, filler, disintegrant, wetting agent, lubricant, colourant, flavourant or other conventional adjuvant or excipient. The term “pharmaceutically acceptable carrier” as used herein is intended to include encapsulating material providing a capsule which surrounds the pharmaceutically active substance per se or together with other pharmaceutically acceptable carriers.

The compound of the present invention, i.e. the Phosphate, and its polymorphic forms A, B, B1, C and E, may be administered by any suitable route, but usually by the oral or parenteral routes.

Pharmaceutical compositions may be prepared by admixture, and are suitably adapted for oral, parenteral or topical administration, and as such may be in the form of tablets, capsules, oral liquid preparations, powders, granules, lozenges, pastilles, reconstitutable powders, injectable and infusable solutions or suspensions, suppositories and transdermal devices.

Suitable methods for formulating the pharmaceutical compositions of the Phosphate, and its polymorphic forms A, B, B1, C and E, are known.

Additionally, the present invention provides a pharmaceutical composition comprising 5-[[4-[2-(methyl-2-pyridinylamino)ethoxy]phenyl]methyl]-2,4-thiazolidinedione phosphate, or one of its polymorphic forms A, B, B1, C and E, or mixtures thereof, in combination with one or more other anti-diabetic agents, e.g. biguanidines, sulfonylureas and alpha glucosidase inhibitors, and optionally with a pharmaceutically acceptable carrier.

In a further aspect, the present invention provides a pharmaceutical composition comprising 5-[[4-[2-(methyl-2-pyridinylamino)ethoxy]phenyl]methyl]-2,4-thiazolidinedione phosphate, or one of its polymorphic forms A, B, B1, C and E, or a mixture thereof, for use as a medicament.

The present invention further provides a method for the treatment and/or prophylaxis of diabetes mellitus, conditions associated with diabetes mellitus and certain complications thereof, in a human or non-human mammal which comprises administering 5-[[4-[2-(methyl-2-pyridinylamino)ethoxy]phenyl]methyl]-2,4-thiazolidinedione phosphate, or one of its polymorphic forms A, B, B1, C and E, or a mixture thereof, to a human or non-human mammal in need thereof. The Phosphate, or its polymorphic forms A, B, B1, C and E, or a mixture thereof, are applied in a pharmaceutically effective, non-toxic, amount. Pharmaceutically effective amounts within the meaning of the present invention include doses that provide a desirable physiological and/or pharmacological effect.

In the treatment and/or prophylaxis of diabetes mellitus, conditions associated with diabetes mellitus and certain complications thereof, the Phosphate, and its polymorphic forms A, B, B1, C and E, may be taken in amounts so as to provide 5-[[4-[2-(methyl-2-pyridinylamino) ethoxy]phenyl]methyl]-2,4-thiazolidinedione in suitable doses, e.g. such as disclosed in EP-A-0306228.

In a further aspect, the present invention provides the use of 5-[[4-[2-(methyl-2-pyridinylamino)ethoxy]phenyl]methyl]-2,4-thiazolidinedione phosphate, and its polymorphic forms A, B, B1, C and E, or a mixture thereof, per se, or comprised in the herein described pharmaceutical compositions, in the for the manufacture of a medicament for the treatment and/or prophylaxis of diabetes mellitus, conditions associated with diabetes mellitus and certain complications thereof.

Additionally, the present invention provides the use of 5-[[4-[2-(methyl-2-pyridinylamino) ethoxy]phenyl]methyl]-2,4-thiazolidinedione phosphate, and its polymorphic forms A, B, B1, C and E, or mixtures thereof, in combination with one or more other anti-diabetic agents, e.g. biguanidines, sulfonylureas and alpha glucosidase inhibitors, for the manufacture of a medicament for the treatment and/or prophylaxis of diabetes mellitus, conditions associated with diabetes mellitus and certain complications thereof.

The following examples illustrate the invention but do not limit it in any way. All temperatures are given in degree Celsius and are uncorrected.

The water content is determined by the Karl Fischer method.

The Infrared absorption spectrum of the herein described polymorphic forms of the Phosphate is measured using a BRUKER FTIR-Tensor 27.

X-Ray Powder Diffraction (XRPD) pattern is measured under the following conditions:

Equipment: X-Ray Powder Diffractometer D-8 (AXS-BRUKER), theta-theta-goniometer, sample changer, target: Copper, Kα1+Kα2 λ=1.5406 Å, parallel beam optics (receiving soller-slit: 0.07 mm), Scintillation counter, standard sample holders.
Data collection: Tube anode: Cu; Generator tension: 40 kV; Generator current: 40 mA; Start angle: 2.0° 2θ, End angle: 40.0° 2θ; Step size: 0.01° 2θ; Time per step: 2 seconds; 2θ may vary 1 to 3% absolutely; 2-theta accuracy of sample data: ±0.05 degrees
Ion chromatography (e.g. for determination of the contents of phosphoric acid) is performed using IC Anion Column SUPER-SEP as available from Metrohm, Switzerland.

### EXAMPLE 1

### Preparation of polymorphic Form A of 5-[[4-[2-(methyl-2-pyridinylamino)ethoxy]phenyl]methyl]-2,4-thiazolidinedione phosphate

5 g of 5-[[4-[2-(methyl-2-pyridinylamino)ethoxy]phenyl]methyl]-2,4-thiazolidinedione are dissolved in a mixture of 250 ml acetone and 20 ml of H₂O at approximately 30° C. The solution is stirred and 1.89 ml of 85% phosphoric acid are added with stirring. Seed crystals of the title compound are added, stirring is stopped and the suspension is allowed to stand at room temperature for about 3 hours with stirring for 2 to 3 minutes in 30 minute intervals. The title compound is isolated by suction, washed with 25 ml of acetone and dried in vacuo for approximately 15 hours at room temperature, and obtained as white crystalline solid. Yield (5-[[4-[2-(methyl-2-pyridinylamino)ethoxy]phenyl]methyl]-2,4-thiazolidinedione phosphate hydrate as polymorphic Form A): 5.54 g

Water content (Karl Fischer): 1.6% w/w

Content Phosphoric acid: 21.7% (by ion chromatography)

### EXAMPLE 2

### Preparation of polymorphic Form A of 5-[[4-[2-(methyl-2-pyridinylamino)ethoxy]phenyl]methyl]-2,4-thiazolidinedione phosphate

25 g of 5-[[4-[2-(methyl-2-pyridinylamino)ethoxy]phenyl]methyl]-2,4-thiazolidinedione are dissolved in a mixture of 1250 ml of acetone and 100 ml of H₂O at approximately 30° C. The solution is stirred, and 9.45 ml of 85% phosphoric acid are added with stirring. Stirring is stopped, and the suspension is allowed to stand at room temperature for about 18 hours. The suspension is then gently stirred for about 1 hour. The white crystals are then isolated by suction, washed with a mixture of 95 ml of acetone and 5 ml H₂O and dried in vacuo for approximately 3 hours at room temperature.

Yield (5-[[4-[2-(methyl-2-pyridinylamino)ethoxy]phenyl]methyl]-2,4-thiazolidinedione phosphate hydrate as polymorphic Form A): 28.56 g

Water content (Karl Fischer): 3.3% w/w

### Characterising Data for the Product of EXAMPLE 2:

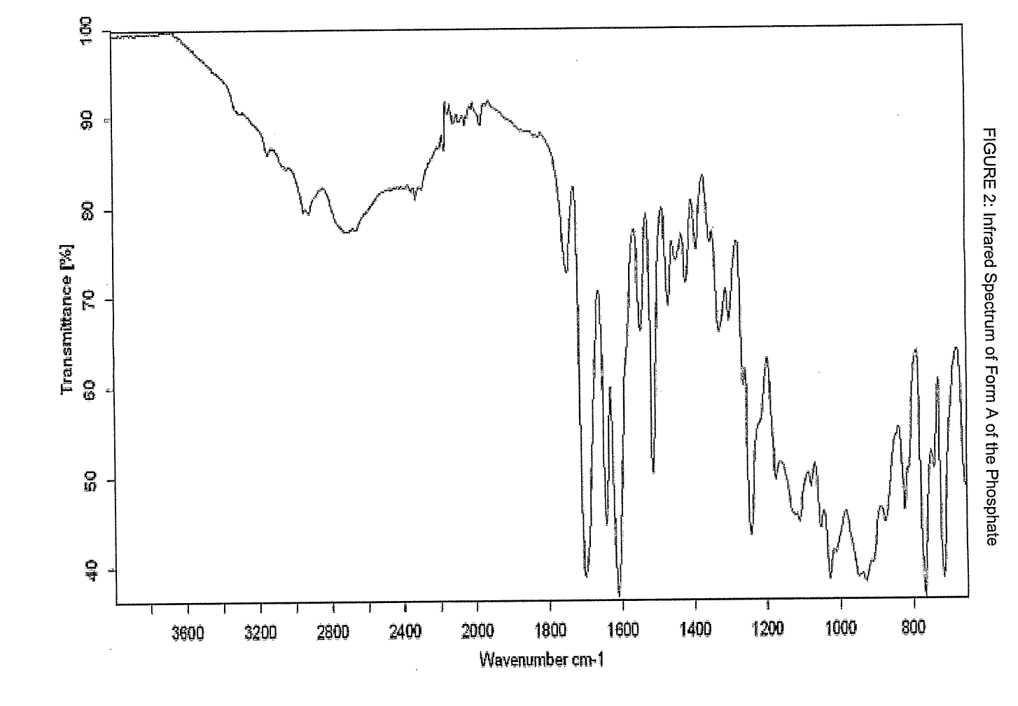

The Infrared absorption spectrum of the solid product as obtained by Example 2 is seen in FIG. 2, and bands observed are as mentioned in the description above.

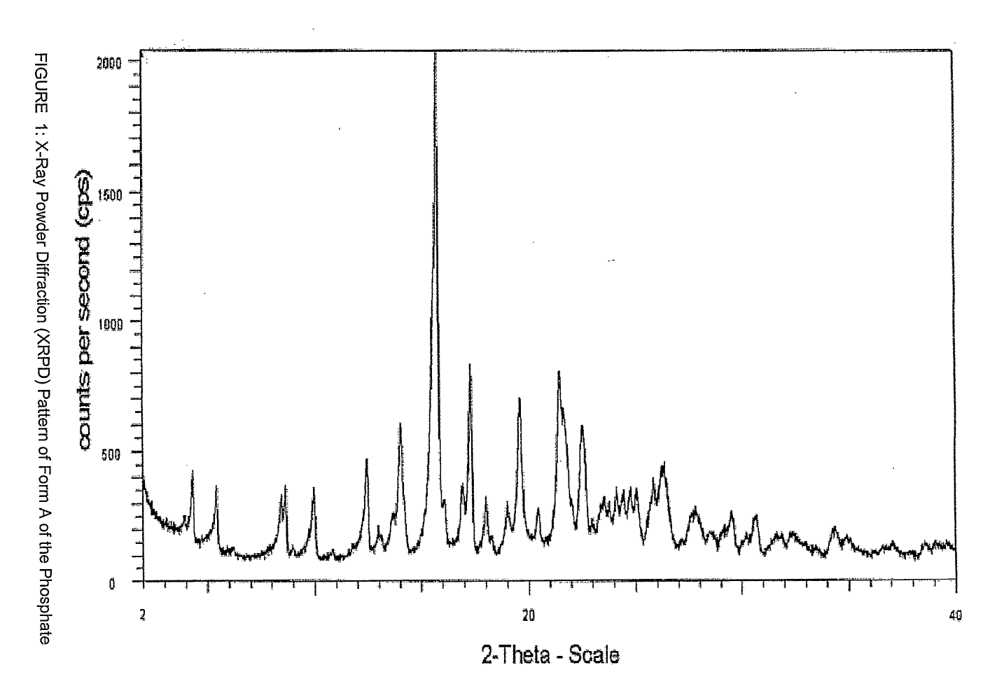

X-Ray Powder Diffraction (XRPD) pattern of the solid product as obtained by Example 2 is shown in FIG. 1, and interplanar spacings (d, given in Å, i.e. Angstroem), characteristic XRPD angles (2 theta°) and relative intensities (in %) are recorded in Table 1.

### EXAMPLE 3

### Preparation of Polymorphic Form A of 5-[[4-[2-(methyl-2-pyridinylamino)ethoxy]phenyl]methyl]-2,4-thiazolidinedione phosphate

10 g of 5-[[4-[2-(methyl-2-pyridinylamino)ethoxy]phenyl]methyl]-2,4-thiazolidinedione are dissolved in a mixture of 500 ml 96% ethanol and 50 ml of H₂O at approximately 60° C. 2.1 ml of 85% phosphoric acid are added. With stirring seed crystals of 5-[[4-[2-(methyl-2-pyridinylamino)ethoxy]phenyl]methyl]-2,4-thiazolidinedione phosphate are added, and the stirring is stopped. The suspension is allowed to stand at room temperature for about 3 hours with stirring for 2 to 3 minutes in 30 minute intervals. The title compound is isolated by suction, washed in 2 portions with a total of 50 ml of ethanol and dried at room temperature in vacuo for about 4 days, and obtained as white crystalline solid.

Yield (5-[[4-[2-(methyl-2-pyridinylamino)ethoxy]phenyl]methyl]-2,4-thiazolidinedione phosphate hydrate as polymorphic Form A): 10.32 g

Water content (Karl Fischer): 2.3% w/w

Content Phosphoric acid: 20.1% (by ion chromatography)

### EXAMPLE 4

### Drying of polymorphic Form A of 5-[[4-[2-(methyl-2-pyridinylamino)ethoxy]phenyl]methyl]-2,4-thiazolidinedione phosphate with phosphorus pentoxide

10 g of 5-[[4-[2-(methyl-2-pyridinylamino)ethoxy]phenyl]methyl]-2,4-thiazolidinedione phosphate hydrate in its polymorphic Form A, water content (Karl Fisher) 2.8% w/w, are dried at a temperature of about 45° C. for about 24 hours in vacuo in presence of P₂O₅.

Water content (Karl Fischer): 0.87% w/w

### EXAMPLE 5

### Exposure of Polymorphic Form A of 5-[[4-[2-(methyl-2-pyridinylamino)ethoxy]phenyl]methyl]-2,4-thiazolidinedione phosphate to humidity

5-[[4-[2-(methyl-2-pyridinylamino)ethoxy]phenyl]methyl]-2,4-thiazolidinedione phosphate hydrate in its polymorphic Form A was exposed to different relative humidities for about 24 hours. Results are given below in Table 7:

TABLE 7

| | Water content (Karl Fisher) |
|---|---|
| | (%) w/w |
| Initial | 2.8 |
| 45% relative humidity | 3.42 |
| 63% relative humidity | 3.37 |
| 86% relative humidity | 3.58 |

### EXAMPLE 6

### Preparation of polymorphic Form A of 5-[[4-[2-(methyl-2-pyridinylamino)ethoxy]phenyl]methyl]-2,4-thiazolidinedione phosphate

61.2 g of 5-[[4-[2-(methyl-2-pyridinylamino) ethoxy]phenyl]methyl]-2,4-thiazolidinedione are dissolved in a mixture of 3060 ml acetone and 244.8 ml water at 30° C. under stirring with aid of a mechanical stirrer. 23.1 ml of 85% phosphoric acid are added. Seeds of Form A are added and the mixture is stirred for about 5 hours at a temperature of about 25° C. The title compound is then isolated by filtration, washed in 2 portions of each 122.4 ml of 95% (v/v) acetone/water and subsequently dried at about 25° C. for approximately 18 hours.

Yield (5-[[4-[2-(methyl-2-pyridinylamino)ethoxy]phenyl]methyl]-2,4-thiazolidinedione phosphate hydrate as polymorphic Form A): 69.1 g

Water content (Karl Fischer): 1.7% w/w

Content Phosphoric acid: 21.2% (by ion chromatography)

### EXAMPLE 7

### Preparation of polymorphic Form B of 5-[[4-[2-(methyl-2-pyridinylamino)ethoxy]phenyl]methyl]-2,4-thiazolidinedione phosphate

20 g of 5-[[4-[2-(methyl-2-pyridinylamino) ethoxy]phenyl]methyl]-2,4-thiazolidinedione are dissolved in a mixture of 1000 ml acetone and 80 ml water at 25-28° C. under stirring with aid of a mechanical stirrer. 4.16 ml of 85% phosphoric acid (1.1 equivalents) are added. Seeds of Form B are added and the mixture is stirred at a temperature of about 25° C. for at least 30 hours. The solid is then isolated by filtration, washed in 2 portions of each 32 ml of 95% (v/v) acetone/water, and is dried at about 40° C. in vacuo for approximately 18 hours to obtain the title compound.

Yield (5-[[4-[2-(methyl-2-pyridinylamino)ethoxy]phenyl]methyl]-2,4-thiazolidinedione phosphate as polymorphic Form B): 25 g (containing traces of B1)

### EXAMPLE 8

### Preparation of polymorphic Form B1 of 5-[[4-[2-(methyl-2-pyridinylamino)ethoxy]phenyl]methyl]-2,4-thiazolidinedione phosphate

20 g of 5-[[4-[2-(methyl-2-pyridinylamino)ethoxy]phenyl]methyl]-2,4-thiazolidinedione are dissolved in a mixture of 1000 ml acetone and 80 ml water at 25-28° C. under stirring with aid of a mechanical stirrer. 4.16 ml of 85% phosphoric acid (1.1 equivalents) are added. Seeds of Form B1 are added and the mixture is stirred at a temperature of about 25° C. for at least 50 hours. The solid is then isolated by filtration, washed in 2 portions of each 32 ml of 95% (v/v) acetone/water and dried at about 40° C. in vacuo for approximately 18 hours to obtain the title compound.

Yield (5-[[4-[2-(methyl-2-pyridinylamino)ethoxy]phenyl]methyl]-2,4-thiazolidinedione phosphate as polymorphic Form B1): approximately 25 g

### EXAMPLE 9

### Preparation of polymorphic Form B1 of 5-[[4-[2-(methyl-2-pyridinylamino)ethoxy]phenyl]methyl]-2,4-thiazolidinedione phosphate

5 g of 5-[[4-[2-(methyl-2-pyridinylamino)ethoxy]phenyl]methyl]-2,4-thiazolidinedione phosphate Form A are suspended in a mixture of 50 ml acetone/water (2:1 v/v). The mixture is stirred alternately for about 1 hour at about 50° C. and subsequently for about 1 hour at about 10° C. all day, at night the mixture is kept at room temperature. The procedure is repeated for a total of about 5 days. The solid is then isolated from the suspension (at 10° C.) by filtration, washed with 10 ml of a mixture acetone/water (95% v/v) and is dried in vacuo for approximately 20 hours to obtain the title compound.

Yield (5-[[4-[2-(methyl-2-pyridinylamino)ethoxy]phenyl]methyl]-2,4-thiazolidinedione phosphate as polymorphic Form B1): approximately 4.3 g

### EXAMPLE 10

### Preparation of polymorphic Form C and conversion to polymorphic Form B of 5-[[4-[2-(methyl-2-pyridinylamino)ethoxy]phenyl]methyl]-2,4-thiazolidinedione phosphate

10 g of 5-[[4-[2-(methyl-2-pyridinylamino) ethoxy]phenyl]methyl]-2,4-thiazolidinedione phosphate Form B are dissolved in a mixture of 50 ml acetone and 50 ml water at about 60° C. With stirring 1.48 ml of 85% of phosphoric acid are added. The suspension is allowed to cool to room temperature and is stirred for about 3 hours. The product, i.e. Form C, is isolated by filtration and washed with a total of 20 ml of acetone/water 95:5 (v/v), and then air dried (open air, relative humidity approximately 28%, at approximately 22° C.) for about 20 hours; subsequently Form C is dried at about 40° C. in vacuo for approximately 20 hours to give Form B.

Yield (5-[[4-[2-(methyl-2-pyridinylamino)ethoxy]phenyl]methyl]-2,4-thiazolidinedione phosphate as polymorphic Form B): 7.02 g

### EXAMPLE 11

### Preparation of polymorphic Form C and conversion to polymorphic Form B1 of 5-[[4-[2-(methyl-2-pyridinylamino)ethoxy]phenyl]methyl]-2,4-thiazolidinedione phosphate

5 g of 5-[[4-[2-(methyl-2-pyridinylamino) ethoxy]phenyl]methyl]-2,4-thiazolidinedione phosphate Form A are suspended in a mixture of 50 ml acetone/water (2:1 v/v). The mixture is stirred alternately for about 1 hour at about 50° C. and subsequently for about 1 hour at about 10° C. all day, at night the mixture is kept at room temperature. The procedure is repeated for a total of about 5 days. The product, i.e. Form C, is then isolated from the suspension (at about 10° C.) by filtration, washed with 10 ml of a mixture acetone/water (95% v/v) and is air dried for approximately 20 hours.

Yield (5-[[4-[2-(methyl-2-pyridinylamino)ethoxy]phenyl]methyl]-2,4-thiazolidinedione phosphate as polymorphic Form C): approximately 4.3 g

Drying at about 40° C. yields Form B1.

### EXAMPLE 12

### Preparation of polymorphic Form D of 5-[[4-[2-(methyl-2-pyridinylamino)ethoxy]phenyl]methyl]-2,4-thiazolidinedione phosphate

10 g of 5-[[4-[2-(methyl-2-pyridinylamino) ethoxy]phenyl]methyl]-2,4-thiazolidinedione phosphate (Form A) are suspended in 150 ml of MeOH (methanol) and the suspension is heated to about 60° C. for about 4 hours. The suspension is getting first rather thin and form D starts to crystallize. The suspension is then stirred for about another 2 hours at room temperature and the title compound is then isolated by filtration, washed with a total of 10 ml of MeOH, and subsequently dried for approximately 20 hours in vacuo.

Yield (5-[[4-[2-(methyl-2-pyridinylamino)ethoxy]phenyl]methyl]-2,4-thiazolidinedione phosphate as polymorphic Form D): 9.72 g

### EXAMPLE 13

### Preparation of polymorphic Form E of 5-[[4-[2-(methyl-2-pyridinylamino)ethoxy]phenyl]methyl]-2,4-thiazolidinedione phosphate

10 g of 5-[[4-[2-(methyl-2-pyridinylamino)ethoxy]phenyl]methyl]-2,4-thiazolidinedione are dissolved in 250 ml of 96% ethanol near the boiling point. The solution is cooled with gentle stirring to about 65° C. and 3.78 ml of 85% phosphoric acid (H₃PO₄) are added. The solution is then allowed to cool to room temperature with gentle stirring with aid of a mechanical stirrer. After addition of phosphoric acid the solution is stirred for the time and at temperatures as follow: about 20 min at about 43° C., about 30 min at about 39° C., about 60 min about 30° C., about 2 hours at about 29° C. and about 22 hours at about 23° C. The title compound is then isolated by filtration, washed in 2 portions with a total of 20 ml of 96% ethanol (EtOH) and dried in vacuo for about 20 hours at about 40° C.

Yield (5-[[4-[2-(methyl-2-pyridinylamino)ethoxy]phenyl]methyl]-2,4-thiazolidinedione phosphate as polymorphic Form E): 12.1 g

Water content (Karl Fischer): 0.2% w/w

The Phosphate, and its polymorphic forms A, B, B1, C, D and E, as herein described, show good stability. After a stress test according to known methods, which was performed at 80° C. for about 160 hours in a closed vial, no degradation has been observed as determined by HPLC using standard methods for Forms A, B, B1 and E.

Furthermore, the present applicants have observed that the 5-[[4-[2-(methyl-2-pyridinylamino)ethoxy]phenyl]methyl]-2,4-thiazolidinedione phosphate, and its polymorphic forms A, B, B1, C, D and E, according to the present invention, exhibit a comparable or even more expressed solubility in water when compared to rosiglitazone maleate which is the main form in which rosiglitazone is currently marketed as active substance in pharmaceutical preparations. Form A, for example, shows an enhanced solubility in water, being e.g. about twice as high as that of the maleate form, which is useful and interesting for industrial application.

Additionally, the processes for the production of the Phosphate, and its polymorphic forms A, B, B1, C, D and E, are relatively simple.

1. A salt of 5-[[4-[2-(methyl-2-pyridinylamino)ethoxy]phenyl]methyl]-2,4-thiazolidinedione and phosphoric acid, or a solvate or a non solvated form thereof.

2. A salt as claimed in claim 1, being 5-[[4-[2-(methyl-2-pyridinylamino)ethoxy]phenyl]methyl]-2,4-thiazolidinedione phosphate wherein the molar ratio of 5-[[4-[2-(methyl-2-pyridinylamino)ethoxy]phenyl]methyl]-2,4-thiazolidinedione to phosphate is 1:1, or a solvate or non-solvated form thereof.

3. A crystalline polymorph A of a salt according to claim 1 or 2 being a 5-[[4-[2-(methyl-2-pyridinylamino)ethoxy]phenyl]methyl]-2,4-thiazolidinedione phosphate hydrate, characterised by an X-ray powder diffraction (XRPD) pattern having intensity peaks at values expressed in 2-theta degrees of about 15.63, 15.75, 17.30, 19.61 and 21.47.

4. A crystalline polymorph A of a salt according to claim 1 or 2 being a 5-[[4-[2-(methyl-2-pyridinylamino)ethoxy]phenyl]methyl]-2,4-thiazolidinedione phosphate hydrate, characterised by an X-ray powder diffraction (XRPD) pattern substantially in accordance with Table 1 and FIG. 1.

5. A crystalline polymorph A according to claim 3 or 4, being a 5-[[4-[2-(methyl-2-pyridinylamino)ethoxy]phenyl]methyl]-2,4-thiazolidinedione phosphate hydrate, characterised by an infrared spectrum with bands observed at 2704, 1748, 1701, 1643, 1611, 1546, 1513, 1469, 1420, 1391, 1330, 1302, 1244, 1110, 1028, 928, 821, 767, 716 cm⁻¹.

6. A crystalline polymorph B of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern having intensity peaks at values expressed in 2-theta degrees of about 4.19, 16.45, 17.01, 18.89 and 21.35.

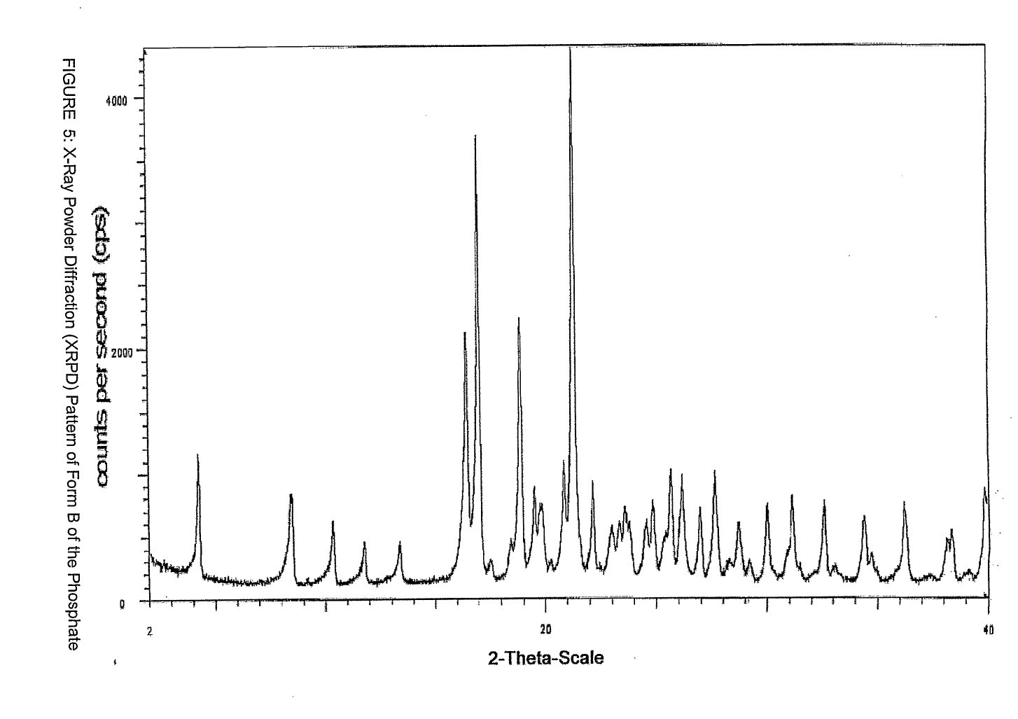

7. A crystalline polymorph B of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern substantially in accordance with Table 3 and FIG. 5.

8. A crystalline polymorph B according to claim 6 or 7, being a 5-[[4-[2-(methyl-2-pyridinylamino)ethoxy]phenyl]methyl]-2,4-thiazolidinedione phosphate, characterised by an infrared spectrum with bands observed at 3050, 2875, 2455, 2325, 2165, 2141, 2114, 2051, 1982, 1874, 1750, 1697, 1640, 1611, 1546, 1513, 1464, 1441, 1416, 13931366, 1333, 1318, 1301, 1284, 1244, 1219, 1181, 1161, 1114, 1097, 1081, 1044, 1030, 994, 948, 924, 896, 826, 812, 772, 741, 712 cm⁻¹.

9. A crystalline polymorph B1 of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern having intensity peaks at values expressed in 2-theta degrees of about 16.46, 19.51, 19.76, 19.88 and 23.31.

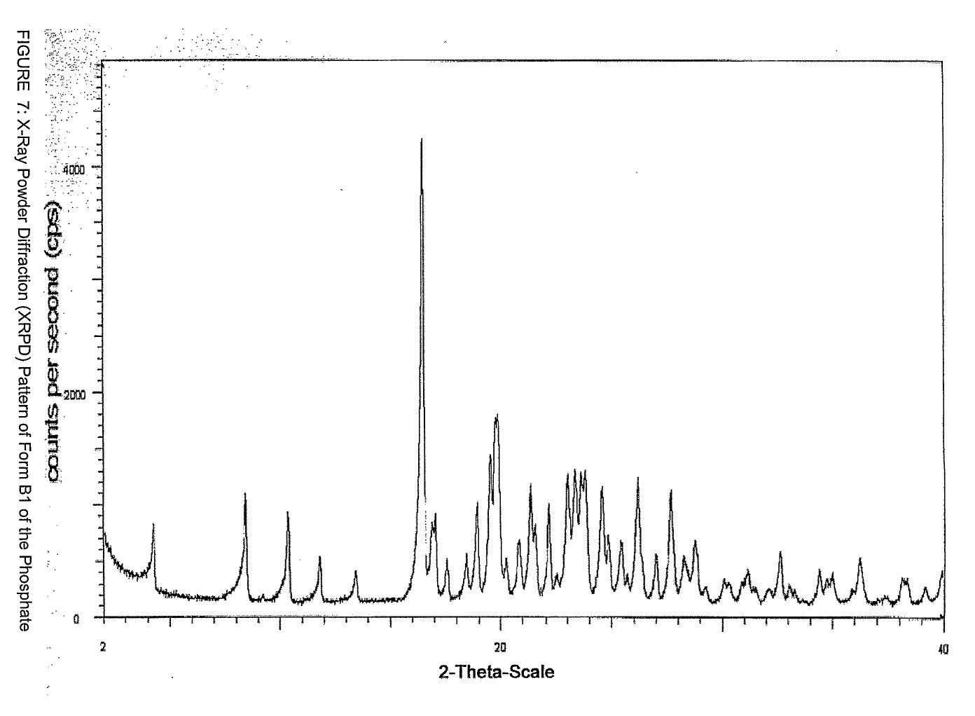

10. A crystalline polymorph B1 of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern substantially in accordance with Table 4 and FIG. 7.

11. A crystalline polymorph B1 according to claim 9 or 10, being a 5-[[4-[2-(methyl-2-pyridinylamino)ethoxy]phenyl]methyl]-2,4-thiazolidinedione phosphate, characterised by an infrared spectrum with bands observed at 3050, 2875, 2455, 2325, 2165, 2141, 2114, 2051, 1982, 1874, 1750, 1697, 1640, 1611, 1546, 1513, 1464, 1441, 1416, 13931366, 1333, 1318, 1301, 1284, 1244, 1219, 1181, 1161, 1114, 1097, 1081, 1044, 1030, 994, 948, 924, 896, 826, 812, 772, 741, 712 cm⁻¹.

12. A crystalline polymorph C of a salt according to claim 1 or 2 being a 5-[[4-[2-(methyl-2-pyridinylamino)ethoxy]phenyl]methyl]-2,4-thiazolidinedione phosphate hydrate characterised by an X-ray powder diffraction (XRPD) pattern having intensity peaks at values expressed in 2-theta degrees of about 12.86, 15.98, 16.26, 21.60 and 24.50.

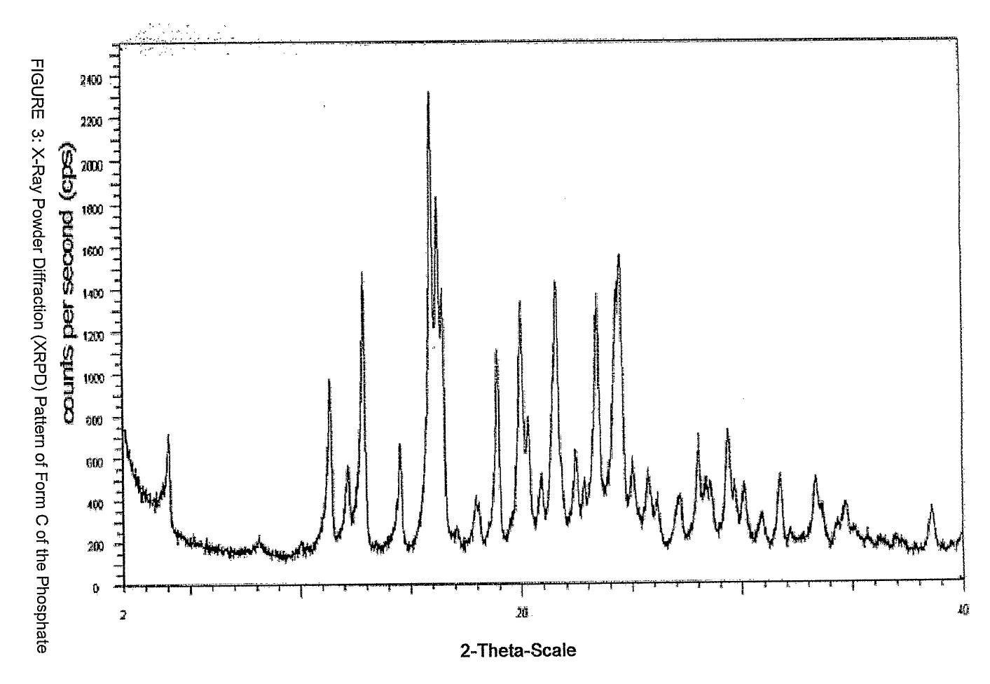

13. A crystalline polymorph C of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern substantially in accordance with Table 2 and FIG. 3.

14. A crystalline polymorph C according to claim 12 or 13, being a 5-[[4-[2-(methyl-2-pyridinylamino)ethoxy]phenyl]methyl]-2,4-thiazolidinedione phosphate, characterised by an infrared spectrum with bands observed at 3111, 2924, 2652, 2325, 2165, 2114, 2051, 1981, 1874, 1745, 1698, 1641, 1608, 1541, 1513, 1464, 1443, 1416, 1392, 1363, 1332, 1301, 1265, 1249, 1218, 1179, 1163, 1113, 1096, 1048, 1028, 995, 951, 926, 905, 823, 812, 774, 739, 713 cm⁻¹.

15. A crystalline polymorph D of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern having intensity peaks at values expressed in 2-theta degrees of about 14.33, 16.05, 16.36, 21.97 and 22.89.

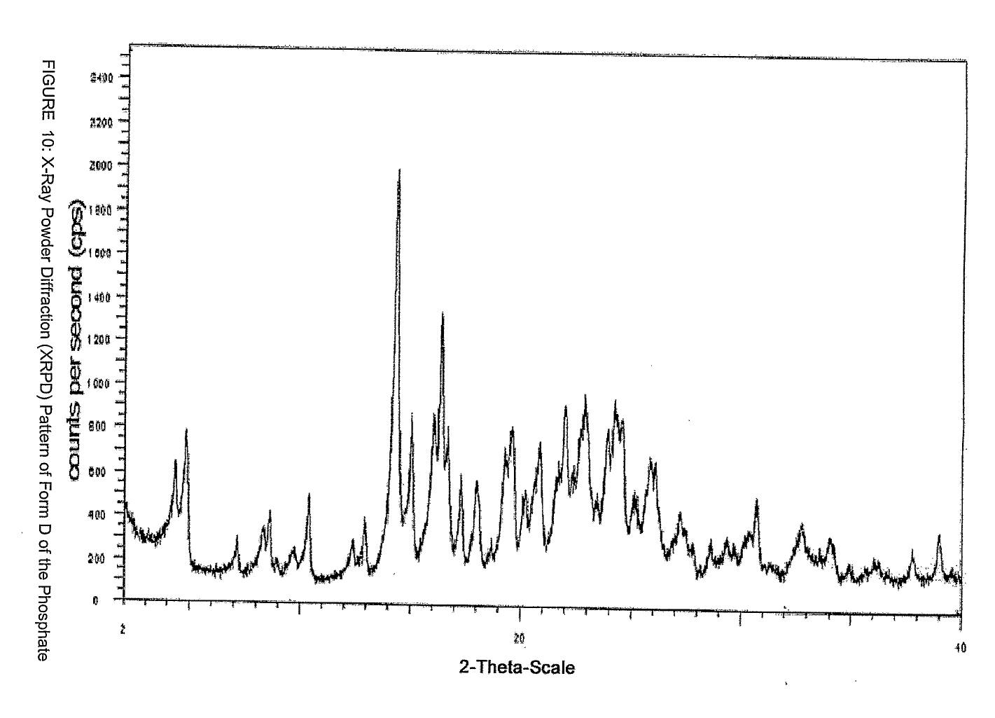

16. A crystalline polymorph D of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern substantially in accordance with Table 6 and FIG. 10.

17. A crystalline polymorph D according to claim 15 or 16, being a 5-[[4-[2-(methyl-2-pyridinylamino)ethoxy]phenyl]methyl]-2,4-thiazolidinedione phosphate, characterised by an infrared spectrum with bands observed at 3129, 2933, 2684, 2325, 2165, 2150, 2113, 2051, 1982, 1743, 1699, 1641, 1604, 1538, 1511, 1467, 1446, 1412, 1389, 1357, 1332, 1303, 1279, 1242, 1164, 1107, 1077, 1063, 1021, 994, 956, 928, 903, 832, 802, 769, 739, 719 cm⁻¹.

18. A crystalline polymorph E of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern having intensity peaks at values expressed in 2-theta degrees of about 4.60, 13.39, 18.20, 18.53 and 22.75.

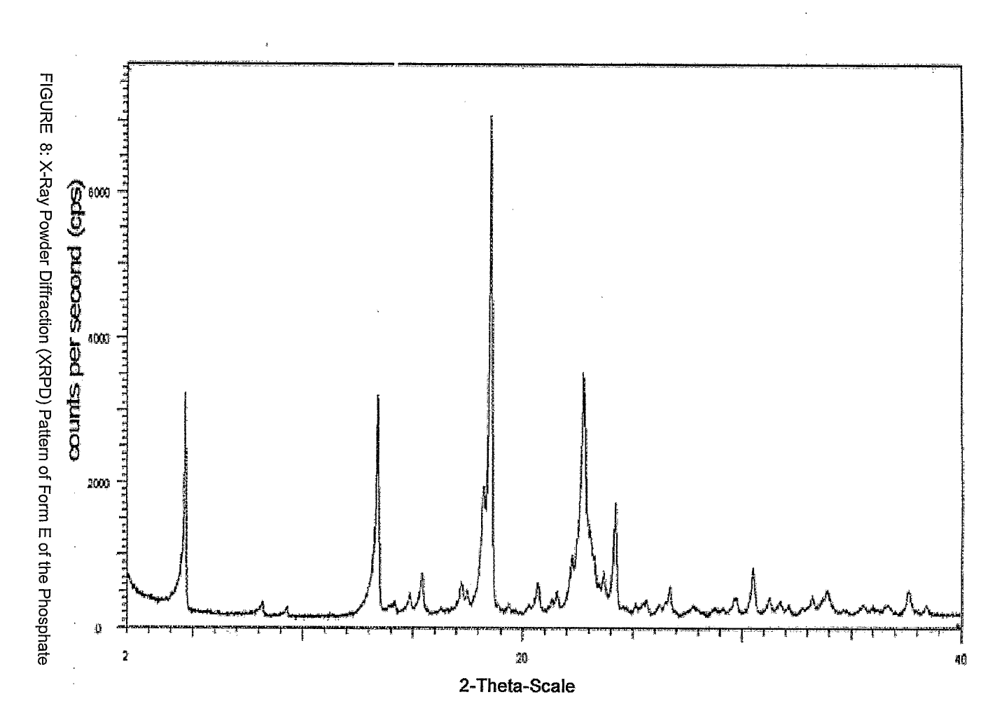

19. A crystalline polymorph E of a salt according to claim 1 or 2 being a 5-[[4-[2-(methyl-2-pyridinylamino)ethoxy]phenyl]methyl]-2,4-thiazolidinedione phosphate characterised by an X-ray powder diffraction (XRPD) pattern substantially in accordance with Table 5 and FIG. 8.

20. A crystalline polymorph E according to claim 18 or 19, being a 5-[[4-[2-(methyl-2-pyridinylamino)ethoxy]phenyl]methyl]-2,4-thiazolidinedione phosphate, characterised by an infrared spectrum with bands observed at 2918, 2702, 2417, 2324, 2165, 2051, 1982, 1752, 1700, 1642, 1610, 1546, 1512, 1468, 1443, 1419, 1395, 1364, 1331, 1303, 1238, 1181, 1165, 1140, 1096, 1052, 1029, 1008, 953, 906, 882, 831, 819, 768, 739, 714, 663 cm⁻¹.

21. A compound according to any one of claims 1 to 20 in isolated form

22. A compound according to any one of claims 1 to 20 in substantially pure form

23. A process for preparing a salt according to claim 1 or 2, comprising reacting 5-[[4-[2-(methyl-2-pyridinylamino)ethoxy]phenyl]methyl]-2,4-thiazolidinedione or a salt thereof, dispersed or suspended or dissolved in a suitable solvent medium, with a suitable source of a phosphate ion.

24. A process for preparing a crystalline polymorph A, B, B1 or E, according to any one of claims 3 to 5, 6 to 8, 9 to 11, or 18 o 20, comprising reacting 5-[[4-[2-(methyl-2-pyridinylamino)ethoxy]phenyl]methyl]-2,4-thiazolidinedione or a salt thereof, dispersed or suspended or dissolved in a suitable solvent medium, with a suitable source of a phosphate ion, and thereafter, carrying out the following steps:
(i) optionally forming a solvate of the 5-[[4-[2-(methyl-2-pyridinylamino) ethoxy]phenyl]methyl]-2,4-thiazolidinedione phosphate,
(ii) recovering the 5-[[4-[2-(methyl-2-pyridinylamino)ethoxy]phenyl]methyl]-2,4-thiazolidinedione phosphate,
(iii) drying the 5-[[4-[2-(methyl-2-pyridinylamino)ethoxy]phenyl]methyl]-2,4-thiazolidinedione phosphate obtained in step ii), especially under vacuum, to obtain the 5-[[4-[2-(methyl-2-pyridinylamino)ethoxy]phenyl]methyl]-2,4-thiazolidinedione phosphate in its polymorphic form A, B, B1 or E.

25. A process according to claim 23 or 24, wherein the suitable source of the phosphate ion is phosphoric acid.

26. A process for preparing a crystalline polymorph C according to any one of claims 12 to 14, comprising the following steps:
(i) dispersing or suspending or dissolving 5-[[4-[2-(methyl-2-pyridinylamino) ethoxy]phenyl]methyl]-2,4-thiazolidinedione phosphate in its polymorphic forms A, B, B1, D or E, in a suitable solvent medium to obtain a mixture,
(ii) stirring the mixture obtained in step (i) alternately for about 1 hour at about 50° C. and subsequently for about 1 hour at about 10° C., for a total of about 3 to about 5 days,
(iii) recovering the product, i.e. polymorph C, from the mixture obtained in step (ii), and
(iv) air-drying the product obtained in step (iii).

27. A process for preparing a crystalline polymorph C according to any one of claims 12 to 14, comprising the following steps:
(i) dissolving or dispersing or suspending 5-[[4-[2-(methyl-2-pyridinylamino) ethoxy]phenyl]methyl]-2,4-thiazolidinedione phosphate of Form A, B, B1, D or E in a suitable solvent medium to obtain a mixture,
(ii) adding a suitable source of a phosphate ion, e.g. phosphoric acid, to the mixture obtained in step (i),
(iii) recovering the product, i.e. polymorph C, from the mixture obtained in step (ii), and
(iv) air-drying the product obtained in step (iii).

28. A process according to claim 26 or 27, wherein the suitable solvent medium is a mixture of acetone and water.

29. A process for preparing crystalline polymorph D according to any one of claims 15 to 17, comprising the following steps:
(i) dissolving or dispersing or suspending 5-[[4-[2-(methyl-2-pyridinylamino) ethoxy]phenyl]methyl]-2,4-thiazolidinedione phosphate in its polymorphic Form A in a suitable solvent medium to obtain a mixture,
(ii) heating the mixture obtained in step (i) to a temperature of about 60° C. for about 4 hours, followed by cooling the mixture to about room temperature under stirring,
(iii) recovering the product, i.e. polymorph D, from the mixture obtained in step (ii), and
(iv) drying the product obtained in step (iii), preferably in vacuo.

30. A process according to claim 29, wherein the suitable solvent medium is methanol.

31. A compound according to any one of claims 1 to 14 and 18 to 20, or a mixture thereof, for use as a medicament.

32. Use of a compound according to any one of claims 1 to 14 and 18 to 20, or of a mixture thereof, for the manufacture of a medicament for the treatment and/or prophylaxis of diabetes mellitus, conditions associated with diabetes mellitus and certain complications thereof in a human or non-human mammal.

33. A pharmaceutical composition comprising a compound according to any one of claims 1 to 14 and 18 to 20, or a mixture thereof, and a pharmaceutically acceptable carrier.

34. A pharmaceutical composition comprising a compound as claimed in any one of claims 1 to 14 and 18 to 20, or a mixture thereof, in combination with one or more other anti-diabetic agents, and a pharmaceutically acceptable carrier.

35. A method for the treatment and/or prophylaxis of diabetes mellitus, conditions associated with diabetes mellitus and certain complications thereof which comprises administering a compound according to any one of claims 1 to 14 and 18 to 20, or a mixture thereof, to a human or non-human mammal in need thereof.

36. A method for the treatment and/or prophylaxis of diabetes mellitus, conditions associated with diabetes mellitus and certain complications thereof which comprises administering a pharmaceutical composition according to claim 33 or 34 to a human or non-human mammal in need thereof.
